(19) United States
(12) Patent Application Publication
(10) Pub. No.: US 2024/0156966 A1
(43) Pub. Date: May 16, 2024

(54) A SIRNA DRUG, A PHARMACEUTICAL COMPOSITION, A SIRNA-SMALL MOLECULE DRUG CONJUGATE, AND THE APPLICATION THEREOF

(71) Applicants: SIRNAOMICS BIOPHARMACEUTICALS (SUZHOU) CO., LTD., Suzhou (CN); SIRNAOMICS BIOPHARMACEUTICALS (GUANGZHOU) CO., LTD., Guangzhou (CN)

(72) Inventors: Patrick Y Lu, Suzhou (CN); Zhiyuan Wang, Suzhou (CN); Shenggao Tang, Guangzhou (CN); Jun Xu, Suzhou (CN); Alan Lu, Suzhou (CN); Guanquan Lin, Guangzhou (CN)

(21) Appl. No.: 18/284,025

(22) PCT Filed: Mar. 25, 2022
(86) PCT No.: PCT/CN2022/083042

§ 371 Date: Sep. 25, 2023

(30) Foreign Application Priority Data
Mar. 26, 2021 (CN) 202110323564.9

(51) Int. Cl. A61K 47/54; A61K 39/00; A61K 39/145; A61K 45/06; C07K 16/10; C12N 15/113

(52) U.S. Cl. CPC A61K 47/549; A61K 39/145; A61K 45/06; C07K 16/1018; C12N 15/1131; C12N 15/1138; A61K 2039/53; C12N 2310/14

(57) ABSTRACT

The present invention relates to a siRNA drug, a pharmaceutical composition, a siRNA-small molecule drug conjugate, and the application thereof. The siRNA molecules block the life cycle of virus replication by targeting and inhibiting the expression of key genes for influenza viruses, reduce viral infection, and ultimately clear the viruses. The present invention provides a drug based on the siRNA. The drug can be atomized into liquid droplets through an atomizing device, and the liquid droplets reach the lower respiratory tract and the lung by inhalation, so as to inhibit the replication of the influenza viruses. The pharmaceutical composition of the present invention can exert a synergistic antiviral effect through different mechanisms of action.

### CROSS-REFERENCE TO RELATED APPLICATIONS

This application is the U.S. National Stage of PCT/CN2022/083042 filed on Mar. 25, 2022, which claims priority to Chinese Patent Application 202110323564.9 filed on Mar. 26, 2021, the entire content of both are incorporated herein by reference in their entirety.

### SEQUENCE LISTING

Incorporated herein by reference is a CRF sequence listing having file name P2338422032ZM-PUS_ST25.txt (47.9 kB), created Sep. 25, 2023.

### FIELD OF THE INVENTION

The present invention belongs to the technical field of biomedicine, and particularly relates to a siRNA drug, a pharmaceutical composition, a siRNA-small molecule drug conjugate, and the application thereof.

### BACKGROUND OF THE INVENTION

Influenza viruses are single-stranded negative-stranded RNA viruses belonging to family Orthomyxoviridae^{[1]}, whose genomes are divided into multiple parts with varying host ranges and pathogenicity. There are influenza A, B, and C viruses (also known as type A, B, and C influenza viruses respectively) that can infect humans, with the influenza A virus being the most virulent. The influenza A virus can infect a variety of avian and mammalian hosts, while the influenza B virus can almost infect humans only. The influenza A virus has drawn widespread concern because it has caused a pandemic. The structure of influenza viruses comprises three parts: a core protein, an envelope protein, and a matrix protein. These proteins are consisted of hemagglutinin (HA), neuraminidase (NA), matrix protein 1 (M1), proton channel protein (M2), nucleoprotein (NP), RNA polymerase (PA, PB1, and PB2), non-structural protein 1 (NS1) and nuclear export protein (NEP, NS2). In addition, some proteins (such as PB1-F2, PB1-N40, and PA-X) were found in certain strains^{[2]}. The influenza A virus is further classified by HA and NA subtypes, with 18 HA subtypes and 11 NA subtypes. For example, H1N1 and H3N2 are human influenza viruses, while H5N1 and H7N9 are avian influenza viruses. HA and NA frequently undergo point mutations (antigenic drift) in seasonal influenza, and gene rearrangement (antigenic transfer) between human viruses and avian viruses may cause a pandemic^{[3]}.

Influenza is a serious problem that affects human health for a long term, the influenza virus infects millions of people every year and causes 250,000 to 500,000 deaths worldwide^{[4]}. Despite the availability of vaccines and antiviral drugs, influenza has serious impacts on health, economy, and society. Due to continuous evolution of the virus, current vaccines can only provide limited protection against influenza. Currently, widespread resistance to adamantanes exists in epidemic viruses, and neuraminidase (NA) inhibitors (NAI) are the only effective antiviral drugs available in most countries. However, NAI is not a perfect solution for influenza viruses, for example, the seasonal influenza A (H1N1) virus circulating around the world in 2008-2009 was resistant to Oseltamivir and had significant toxic and side effects^{[5]}. Overall, influenza virus infection remains a threat to human health and society. Therefore, the development and clinical application of novel antiviral drugs with different mechanisms of action are crucial.

At present, the drugs that have been marketed against influenza A virus infection include: viral neuraminidase (NA) inhibitors Relenza (Zanamivir), Tamiflu (Oseltamivir phosphate), Inavir (Laninamivir octanoate), and Rapivab (Peramivir); M2 ion channel blockers Amantadine and Rimantadine; a viral polymerase inhibitor Favipiravir; and broad-spectrum antiviral drugs Ribavirin and Arbidol. However, each of the above-mentioned drugs has various limitations. For example, the viral neuraminidase inhibitors still face some problems, such as oral efficacy, drug resistance, and induction of cytokine storms. The long-term, extensive, and massive use of adamantanes has resulted in severe drug resistance in most influenza A viruses^{[6]}. The adverse reaction data of Favipiravir is incomplete, and there is also the problem of drug resistance^{[7]}.

Among various antiviral drugs that can be used to treat influenza infection, one of the most commonly used drugs is Oseltamivir (Tamiflu®)^{[8]}. Although the mechanism of action of Oseltamivir as the neuraminidase inhibitor is well known, the effect of Oseltamivir on the dynamics of human influenza viruses has been controversial. Pharmaceutical companies such as Roche have conducted many clinical trials of Oseltamivir, but until recently, data from some early clinical trials was available as pdf scans of edited reports, rather than as actual raw data, and therefore the data could not be analyzed in more detail by other researchers. Typically, such reports include median viral shedding curves for influenza virus infection treated with placebos and drugs, often indicating high efficacy of early treatment. However, median shedding curves may not accurately represent an individual's impact on a drug.

In past influenza studies, the more previous PB1 transcriptase inhibitor Ribavirin has been administered orally, in the form of an aerosol or intravenously, but has not yet shown convincing clinical efficacy^{[9]}. In a double-blind randomized controlled trial (RCT), the combination of oral Amantadine, Ribavirin, and Oseltamivir (called triple antiviral drug or TCAD) was tested. In preclinical models (including models using the virus), the efficacy of the triple antiviral drugs is better than that of single or dual drugs, including the efficacy against Amantadine-resistant strains. Outpatients with high risk of influenza complications within 5 days after the onset of symptoms were randomly assigned to receive TCAD (oral administration of Oseltamivir 75 mg, Amantadine 100 mg, and Ribavirin 600 mg) or Oseltamivir twice a day (BID)^{[10]}. In 394 patients with confirmed influenza virus infection, TCAD has significantly greater antiviral effects than Oseltamivir alone (on the third day, viral RNA can be detected in 40.0% of patients treated with the TCAD, while viral RNA can be detected in 50.0% of patients treated with the Oseltamivir alone). However, the effects on some diseases are not very good, which may be related to the side effects of the TCAD regimen. The TCAD group had a higher proportion of serious adverse events and hospitalization. Therefore, compared with the Oseltamivir alone, the triple-drug regimen failed to improve the clinical efficacy in outpatients with increased risk of influenza complications.

Favipiravir (T-705) was first developed in Japan, and was approved for influenza pandemic prevention in Japan on Mar. 24, 2014. Since Favipiravir increases the risks of teratogenicity and embryotoxicity, only a conditional marketing authorization has been obtained, and strict regulations have been established for its production and clinical use^{[11]}. Therefore, Favipiravir is only applicable to patients infected with novel or recurrent pandemic influenza viruses, on whom other influenza antiviral drugs are ineffective or insufficiently effective. In other countries/regions, Favipiravir is still in the stage of clinical research. Favipiravir is phosphorylated in infected cells, converted to its active form, recognized by RNA-dependent RNA polymerase (RdRP) as a purine analogue, and efficiently integrated into newly formed RNA strands as guanosine and adenosine analogues^{[12]}.

Nucleic acid interference (RNAi) technology was first discovered in nematodes, and at that time, double-stranded RNA (dsRNA) complementary to specific genes was found to be more effective than single-stranded RNA in silencing the expression of corresponding genes^{[13]}. Subsequent studies have shown that the silencing effect is related to 21-23 bp nucleotide double-stranded small interfering RNA (siRNA) and dsRNA specific endoribonuclease III (called Dicer), and the latter is responsible for cleaving dsRNAs into siRNAs, thereby triggering the silencing mechanisms of RNAi. Specifically, siRNA binds to specific proteins/enzymes to form RNA-induced silencing complexes (RISCs), where the sense strand in siRNA detaches and the antisense strand targets sequence-specific mRNA and binds to it^{[14]}. Subsequently, enzymes in RISC cleave the target mRNA by about 12 nucleotides from the 3′ end of the siRNA strand, resulting in mRNA degradation and gene silencing.

The RNAi technology has become a powerful tool for antiviral infection, and siRNA has many advantages compared with small molecule chemical drugs^{[15]}. First, siRNA “drugs” can be rapidly synthesized and scaled up for production. Second, in the case of viral resistance to one siRNA, different siRNAs targeting another viral sequence can be used, or even two or more siRNA molecules targeting different genes of the influenza viruses can be used simultaneously. Third, regardless of the siRNA sequence, all siRNAs use the same synthetic chemistry methods, so that it is very easy to combine the two siRNAs together using the same manufacturing process. In addition, unlike many organic compounds with pharmacological activity, the siRNA is water-soluble, which is very beneficial for drug utilization.

The siRNA also showed good effects when being used for resisting influenza A virus infection. In a study using MDCK cells, it was observed that the siRNA had a significant inhibitory effect on H1N1 viruses, and the specific siRNA could inhibit the expression of influenza virus mRNA by about 50%. Plaque assay showed that siRNA could reduce the influenza viral titer to 1/200^{[16]}. The researchers extended the length of the siRNA molecule form the designed 19 bp to 27 bp, indicating that increasing the length of the siRNA can inhibit HIN1 and H3N2 multiple strains by more than 60%, and the inhibition effect can be most obvious at 48 h^{[17]}. The siRNA can also inhibit proliferation of influenza viruses in animals and improve the protection rate for the animals. In one study, the siRNA was intravenously injected into mice, and viral titers decreased after H1N1 challenge. Then, 18 days after a single injection of one or two siRNAs into mice, the protection rate of the single siRNA was 80%-90%, while the survival rate of the mice treated with two siRNAs reached 100%^{[18]}. In another study, the siRNA was injected into the nasal cavity and intravenously injected into the mice concurrently, and it was found that mRNA and proteins were inhibited by more than 90%, and inflammatory factors were also significantly reduced^{[19]}.

However, it is well known that influenza viruses have the ability to mutate rapidly and can easily generate drug resistance through mutation. There are two mechanisms for the variability of influenza virus antigens: (1) the viral HA and NA genes are prone to mutation (antigenic drift), resulting in the formation of new antigens (thus avoiding previously existing host immunity), and the main cause of drift is that viral polymerases are prone to errors; and (2) rearrangement of gene segments between two different influenza viruses in the same host causes antigenic transfer, resulting in new virus strains. Some researchers believe that the influenza A (H1N1) pandemic in 1918 was caused by recombinant viruses between human influenza virus strains and avian influenza virus strains; and similarly, the “swine influenza” A H1N1 influenza pandemic that have broken out continuously in recent years was also caused by a series of recombination events among human influenza A H3N2, swine influenza H1N1, and avian influenza H1N2^{[20]}.

The viruses are resistant to drugs due to the rapid mutation ability of the influenza viruses, and therefore the combination therapy of two or more drugs has become an effective way to deal with the influenza viruses. In vitro cell^{[21]} and mouse experiments^{[22-23]} have demonstrated the synergistic effect of Favipiravir and NA inhibitors. A phase IIa trial of Favipiravir suggested that the combination therapy of Favipiravir and Oseltamivir may accelerate clinical recovery in hospitalized patients aged 18 years and above with severe influenza^{[24]}. However, the combined use of various small molecule compounds cannot achieve a very significant synergistic effect due to the similarities in the structure, mechanism of action, bioavailability, and half-life of the drugs.

### SUMMARY OF THE INVENTION

The purpose of the present invention is to provide a siRNA molecule for efficiently and specifically inhibiting replication of the influenza virus and a new siRNA drug, pharmaceutical composition, and siRNA-small molecule drug conjugate for preventing and treating influenza virus infection.

To achieve the above purpose, the technical protocol adopted in the present invention is:

A first aspect of the present invention is to provide a siRNA molecule for inhibiting replication of the influenza virus. The siRNA molecule includes a sense strand and an antisense strand, and the sequence of the sense strand is selected from any one of SEQ ID No. 1-16, SEQ ID No. 20-54, SEQ ID No. 56-69, SEQ ID No. 71-91, SEQ ID No. 93, and SEQ ID No. 94, and the antisense strand is selected from any one of SEQ ID No. 98-113, SEQ ID No. 117-151, SEQ ID No. 153-166, SEQ ID No. 168-188, SEQ ID No. 190, and SEQ ID No. 191, which is complementary to the sense strand.

A second aspect of the present invention is to provide a siRNA drug for preventing or treating influenza virus infection. The siRNA drug includes active ingredient(s), and the active ingredient(s) are one or more of the siRNA molecules of claim 1.

The active ingredient(s) may further include one or more other siRNA molecules for inhibiting replication of the influenza virus.

Further, the sequence of the sense strand of the other siRNA molecules for inhibiting replication of the influenza virus may be selected from any one of SEQ ID No. 17-19, SEQ ID No. 55, SEQ ID No. 70, SEQ ID No. 92, SEQ ID No. 95-97, and the antisense strand of other siRNA molecules for inhibiting replication of the influenza virus may be selected from any one of SEQ ID No. 114-116, SEQ ID No. 152, SEQ ID No. 167, SEQ ID No. 189, and SEQ ID No. 192-194, which is complementary to the sense strands of the other siRNA molecules for inhibiting replication of the influenza virus.

A third aspect of the present invention is to provide a pharmaceutical composition for preventing or treating influenza virus infection, and the active ingredients of the pharmaceutical composition include a siRNA molecule for inhibiting replication of the influenza virus and the other molecule(s), and the other molecule(s) include one or more of a siRNA molecule for inhibiting PD-1 expression, a siRNA molecule for inhibiting the expression of PD-L1, anti-influenza virus small molecule compound, an influenza mRNA vaccine, or a monoclonal antibody for resisting the influenza viruses.

The siRNA molecules for inhibiting replication of the influenza virus are designed for conserved gene sequences for different strains of influenza A virus, including one or more of H1N1, H5N1, H7N9, or H3N2 subtypes; the siRNA molecules for inhibiting replication of the influenza virus block the life cycle of virus replication by targeting and inhibiting the expression of key genes related to invasion, replication, assembly or release of the influenza virus, reduce viral titers, and inhibit infection until complete virus clearance.

The siRNA molecule(s) for inhibiting replication of the influenza virus may be selected from one or more of the following siRNA molecules: any one of SEQ ID No. 1-97, and the antisense strand may be selected from any one of SEQ ID No. 98-194, which is complementary to the sense strand.

The siRNA molecule for inhibiting PD-1 expression is designed based on the homologous sequences between a human PD-1 gene and a mouse PD-1 gene, and the siRNA molecule for inhibiting PD-L1 expression is designed based on the homologous sequences between a human PD-L1 gene and a mouse PD-L1 gene.

Further, the homologous sequences refer to the DNA sequences confirmed to be 100% identical after the two genes of humans and mice are aligned.

siRNA molecules targeting PD-L1 are small interfering nucleotides that specifically inhibit the expression of human programmed death factors 1 (PD1) and ligands 1 (PD-L1). The PD-L1 has significant impacts on the immune system of the body, and can inhibit the function of T cells during virus infection, or cause T cell exhaustion.

According to some embodiments, the siRNA molecule(s) for inhibiting PD-1 expression are selected from one or more of the following siRNA molecules: the sequence of the sense strand is selected from any one of SEQ ID No. 195-206, and the antisense strand is selected from any one of SEQ ID No. 207-218, which is complementary to the sense strand.

According to some embodiments, the siRNA(s) for inhibiting PD-L1 expression are selected from one or more of the following siRNA molecules: the sequence of the sense strand is selected from any one of SEQ ID No. 219-230, and the antisense strand is selected from any one of SEQ ID No. 231-242, which is complementary to the sense strand.

The influenza mRNA vaccine is a messenger ribonucleic acid vaccine designed according to the influenza virus gene sequence.

Further, the influenza virus gene may be a gene encoding a viral structural protein and/or a non-structural protein. Further, the gene encoding the viral structural protein(s) may be selected from one or more of PB2, PB1, PA, HA, NP, NA, M1, or M2, and the gene(s) encoding the non-structural protein may be NS1 and/or NS2.

The mRNA vaccine may not only contain specific viral gene sequences, but also elements necessary for translation in cells.

Further, the elements may include, but are not limited to, untranslated regions (UTR) at both ends, cap structures at the 3′ end and polyA tails at the 5′ end.

The anti-influenza virus small molecule compound may be a specific influenza virus inhibitor and/or a broad-spectrum antiviral small molecule compound.

Further, the specific influenza virus inhibitor(s) may be selected from one or more of M2 ion channel blockers, NA (neuraminidase) inhibitors, PA (polymerase PA subunit) inhibitors, and PB2 (polymerase PB2 subunit) inhibitors.

Further, the broad-spectrum antiviral small molecule compound(s) may be selected from one or more of Ribavirin, Nitazoxanide, Arbidol hydrochloride, and Favipiravir.

The small molecule compound may be a water-soluble compound.

Further, the small molecule compound may have good stability after being dissolved in an aqueous solution, and can still maintain certain activity after being atomized.

A fourth aspect of the present invention is to provide a siRNA-small molecule drug conjugate, and the siRNA-small molecule drug conjugate is formed by covalent bond coupling of the siRNA molecule for inhibiting replication of the influenza virus and the anti-influenza small molecule drug.

The small molecule compound may be a small molecule containing a nucleotide base structure.

The chemical bond may be a covalent bond, an ionic bond or a metallic bond.

The siRNA molecules for inhibiting replication of the influenza virus are designed for conserved gene sequences for different strains of influenza A virus, including one or more of H1N1, H5N1, H7N9, or H3N2 subtypes; the siRNA molecules for inhibiting replication of the influenza virus block the life cycle of virus replication by targeting and inhibiting the expression of key genes related to invasion, replication, assembly or release of the influenza virus, reduce viral titers, and inhibit infection until complete virus clearance.

The siRNA molecule for inhibiting replication of the influenza virus may be selected from any one of SEQ ID No. 1-97, and the antisense strand is selected from any one of SEQ ID No. 98-194, which is complementary to the sense strand.

The anti-influenza virus small molecule compound may be a specific influenza virus inhibitor and/or a broad-spectrum antiviral small molecule compound.

Further, the specific influenza virus inhibitor(s) may be selected from one or more of M2 ion channel blockers, NA inhibitors, PA inhibitors, and PB2 inhibitors.

Further, the broad-spectrum antiviral small molecule compound(s) may be selected from one or more of Ribavirin, Nitazoxanide, Arbidol hydrochloride, and Favipiravir.

The siRNA molecule for inhibiting replication of the influenza virus may be linked to the anti-influenza virus small molecule compound through respective active groups, or is coupled with the anti-influenza virus small molecule compound through the active groups of the Linker by introducing Linker into the siRNA molecule for inhibiting replication of the influenza virus.

The active group(s) may include one or more of amino groups, carboxyl groups, hydroxyl groups, phosphate groups, epoxy groups, aldehyde groups, and isocyanate groups.

A fifth aspect of the present invention is to provide an application of the siRNA-small molecule drug conjugate to the preparation of a drug for preventing or treating influenza virus infection.

The siRNA drug for preventing or treating influenza virus infection, the pharmaceutical composition for preventing or treating influenza virus infection, or the siRNA-small molecule drug conjugate forms a formulation with pharmaceutically acceptable carrier(s), and the pharmaceutically acceptable carrier(s) are selected from one or more of saline water, saccharides, polypeptides, high molecular polymers, lipids, creams, gels, micellar materials, or metal nanoparticles.

Further, the high molecular polymers may be polypeptide-based high molecular polymers.

Still further, the polypeptide-based high molecular polymers may be cationic polypeptides consisting of histidine and lysine.

According to some specific embodiments, the polypeptide-based high molecular polymers are HKP (H3K4b) and/or HKP (+H) branched polypeptides.

Still further, the siRNA drug, the pharmaceutical composition, or the siRNA-small molecule drug conjugate may form a nano-formulation with the pharmaceutically acceptable carrier(s); and the nano-formulation is an oral formulation, injection, or aerosol inhalation formulation.

According to some specific embodiments, the dosage form of the nano-formulation is an aerosol inhalation formulation, and the formulation is delivered to disease sites by intravenous injection, oral administration, subcutaneous injection, intramuscular injection, aerosol inhalation, intranasal administration and other manners to exert inhibitory effect on viruses.

After the nano-formulation is atomized by an ultrasonic atomization device, the drug is delivered to the lower respiratory tract and the lung by inhalation to inhibit replication of the influenza virus.

The siRNA drug for preventing or treating influenza virus infection, or the pharmaceutical composition for preventing or treating influenza virus infection, or the application according to the present invention, is directed against one or more influenza virus(es) of G4 EA H1N1 virus strains, H1N1 virus strains, H5N1 virus strains, H7N9 virus strains, or H3N2 virus strains.

The present invention combines the siRNA molecules with other types of anti-influenza virus drugs based on siRNA molecules for inhibiting replication of the influenza virus. This combination strategy aims to provide an effective and complementary strategy for the treatment of influenza virus infection and is more effective than each therapy alone. Each siRNA proven to have a significant inhibitory effect on the influenza virus is combined with an anti-influenza virus small molecule drug which has been marketed or clinically validated, and the broad-spectrum efficacy against the influenza virus and other infection models in cells, rodents, and non-human primates is evaluated.

The pharmaceutical composition of the present invention can be a combination of the siRNA molecule for inhibiting replication of the influenza virus and the other molecule in a specific ratio and it can be administered in the same manner as a mixed solution. The specific ratio is determined according to the concentrations required for the two molecules to exert efficacy, especially the plasma concentration. For the siRNA molecule for inhibiting replication of the influenza virus, the drug concentration is determined according to the data results of preclinical studies. For the other molecule, the drug concentration is determined according to the preclinical data, and data from clinical trials and clinical applications. For the composition obtained by mixing in the specific ratio, the synergistic effect and interaction between the drugs are also considered.

The pharmaceutical composition of the present invention can be used as a combination of solutions of the siRNA molecule for inhibiting replication of the influenza virus as a single drug solution and the other molecule as the other single drug solution. The two single drug solutions are dissolved in the same or similar solvent. The two single drug solutions are dissolved in different solvents. The two single drug solutions in the composition can be administered simultaneously or at different times. Further preferably, the two single drug solutions are administered sequentially at approximately the same time, or are administered alternately at different times.

In the present invention, the siRNA drug molecule, the pharmaceutical composition, and the siRNA-small molecule drug conjugate can also be combined with a pharmaceutically acceptable nano-introduction carrier conjugate to form a nano-drug. The nano-drug carrier is combined with various molecules through electrostatic interaction, hydrogen bonds and Van der Waals force to form a stable uncoupled nano-polymer. The nano-introduction carrier can encapsulate the siRNA molecule for inhibiting replication of the influenza virus and the other molecule at the same time, or encapsulate the two molecules separately. The nano-introduction carrier encapsulates the two molecules at the same time to form nano-drug particles with uniform particle size. When the two molecules are separately encapsulated, the identical nano-introduction carriers can be used to encapsulate the two molecules respectively to form nanoparticles with the same or different particle sizes, or different nano-introduction carriers can also be used to encapsulate the two molecules respectively to form nanoparticles with the same or different particle sizes.

The nano-drug particles are polymers that stably suspend in the form of particles in the specific solvent, and the diameters of the nano-drug particles range from a few nanometers to hundreds or even thousands of nanometers. Preferably, the diameter of the nanoparticles is 30-300 nanometers, and further preferably, the size of the nanoparticles is 50-150 nanometers. The nano-drug can be administered by aerosol inhalation, intravenous injection, subcutaneous injection, intramuscular injection, oral administration and the like. The nano-drug can be atomized into droplets by an ultrasonic atomization device, and administered by inhalation to reach the lower respiratory tract and the lung to inhibit replication of the influenza virus. When the nanoparticles are prepared from the two molecules separately, the two nanoparticles can be administered in the same way or in different ways.

Due to the application of the above technical protocol, the present invention has the following advantages compared with the current technology:

The present invention provides the siRNA molecule for efficiently and specifically inhibiting replication of the influenza virus, which has a significant inhibitory effect on the influenza virus, and provides more options for preparing drugs for preventing or treating influenza virus infection. Based on the siRNA molecule for inhibiting replication of the influenza virus, more new siRNA drugs for preventing and treating influenza virus infection can be prepared, or the siRNA molecule for inhibiting replication of the influenza virus can be combined with other types of anti-influenza virus drugs to prepare the pharmaceutical composition and the siRNA-small molecule drug conjugate based on the siRNA molecule, so as to provide effective and complementary strategies for treating influenza virus infection and broad-spectrum efficacy in infection models such as influenza viruses in cells, rodents, and non-human primates.

### BRIEF DESCRIPTION OF THE DRAWINGS

FIG. 1. An anti-influenza virus siRNA molecule can effectively inhibit replication of the virus in in-vitro cell experiments. A shows the titers of H5N1 influenza virus hemagglutinin (HA) in the cell supernatant after different treatments, B shows TCID₅₀ (median tissue culture infective dose) of an H5N1 virus in the cell supernatant after different treatments, and a siRNA sequence (80 nM) of M1-1 has the best inhibitory effect on an H5N1 influenza virus. C shows the titer of H1N1 influenza virus hemagglutinin (HA) in the cell supernatant after different treatments, and D shows the TCID₅₀ (median tissue culture infective dose) of an H7N9 virus in the cell supernatant after different treatments. It can be seen that siRNA molecules such as M1-1, PA-19, and NP-15 can effectively inhibit H1N1 virus infection, and the siRNA sequence (80 nM) of M1-1 has a significant inhibitory effect on H7N9;

FIG. 2. Anti-influenza virus siRNA molecules reduce the death of mice caused by influenza virus infection. A shows animal grouping, and administration dosage and mode, and B shows the survival curve of the mice in each group. It can be seen from the Figs that the medium dose of M1-1 siRNA intravenous injection group has a good inhibitory effect on the viruses. The survival rate (70%) of the mice on the 15 h day is significantly higher than that of the negative siRNA control group, and also higher than that of the Tamiflu (Oseltamivir in the Fig.) treatment group;

FIG. 3. Expression rates of the PD-1 or PD-L1 gene in different cells after siRNA transfection. A and B show the expression rates of target genes PD-1 and PD-L1 after siRNA against PD-1 and PD-L1 genes was transfected with MCF-7 (breast cancer) cells, BxPC3 (pancreatic cancer) cells, and HepG2 (liver cancer) cells;

FIG. 4. Inhibition of PD-1 expression by siRNA can activate immune cells to secrete cytokines. After transfection of mouse RAW264.7 macrophages with siRNA against PD-1, the concentration of TNF-α in the cell culture supernatant was significantly increased (see FIG. 4A), and the concentration of TNF-α in cell lysate was also increased (no statistically significant differences, see FIG. 4B);

FIG. 5. Schematic diagram of combination of anti-influenza virus siRNA and mRNA vaccines against influenza A virus infection. The mRNA vaccine can be expressed into specific protein/peptide fragments of influenza viruses in the body, to activate the immune mechanism against influenza virus infection, and reduce the probability of virus infection. At the same time, siRNA is used for inhibiting the replication of the viruses in the cells and blocking the life cycle of the viruses;

FIG. 6. Conjugation of Zanamivir to siRNA molecules. The Zanamivir is linked to the end of the siRNA by the condensation of phosphate groups and hydroxyl groups into phosphate esters, or by Linker;

FIG. 7. Conjugation of Peramivir to the siRNA molecules. The carboxyl group of the Peramivir can be linked to the siRNA through Linker;

FIG. 8. Conjugation of Oseltamivir to the siRNA molecules. The hydroxyl group at one end of Linker reacts with the phosphate group at the end of the siRNA, and the epoxy group at the other end is subjected to a nucleophilic reaction with the amino group of Oseltamivir;

FIG. 9. Conjugation of A-192558 and A-315675 to the siRNA molecules. A-192558 contains modifiable amino groups and carboxyl groups. It can be linked to the siRNA by reaction of the epoxy group at one end of Linker with the amino groups and reaction of the hydroxyl group at the other end of the Linker with the phosphate group of siRNA; or by addition reaction with carbon-carbon double bonds through hydrogen halides and linkage to the Linker by substitution reactions;

FIG. 10. Aerosol inhalation (short-duration and short-interval) administration can efficiently deliver the siRNA molecules to the lung. A shows the distribution of the siRNA molecules in the lung after aerosol inhalation, and B shows the inhibitory effect of the siRNA molecules on the target genes of the lung;

FIG. 11. Aerosol inhalation (long-duration and long-interval) administration can effectively deliver the siRNA molecules to the lung. A shows the distribution of the siRNA molecules in the lung after aerosol inhalation, and B shows the distribution of the siRNA molecules in the lung 24 hours after aerosol inhalation; and

FIG. 12. The aerosol inhalation administration of siRNA has no significant toxic and side effects. A: aerosol inhalation administration has no significant effects on the body weight of the mice; B: pulmonary interleukin 6 (IL-6) has no significant changes after aerosol inhalation administration; and C: pulmonary TNF-α has no significant changes after aerosol inhalation.

### DETAILED DESCRIPTION OF EMBODIMENTS

### Embodiment 1. siRNA Molecules for Efficiently Inhibiting Virus Infection

MDCK was cultured in a medium containing 10% MEM, and subjected to amplification culture at a ratio of 1:1 to maintain cell viability. 12-18 hours before transfection, MDCK cells were added to a 24-well plate at 2.4×10⁵ cells/well, and transfection was performed when the cell density reached about 80%. Transfection follows the instructions of Lipofectamine 2000 Liposome Transfection Reagent (Life Technologies). Oseltamivir control drugs were added directly to the medium and the final concentration is 125 μM. 24 hours after transfection (or adding Oseltamivir), a cell culture plate was washed with PBS three times, and then OPTI-MEM of 400 μL/well was added. After the virus infection dose (selected MOI=0.01) was optimized, the viruses were diluted with OPTI-MEM, and inoculated with 100 μL/well. The treated cell culture plate was placed in a cell culture incubator for adsorption for 1 h, during which the cell plate was gently shaken every 15 min. After 1 hour, the virus solution was sucked out and discarded, after the cell plate was washed with PBS, the contents in the cell plate were replaced with 1 mL of OPTI-MEM which contains 0.5% of antibiotics in each well, and the cell plate was cultured in a 37° C. cell culture incubator for 3 days. 48 hours after virus inoculation, the cell supernatant was collected at 200 Lt/well for determining HA titers and TCID₅₀ (median tissue culture infective dose). The HA titers were determined by conventional methods. A method for determining the TCID₅₀ comprises the following steps: each sample was diluted with 1% MEM by 8 dilutions, and for each dilution, the diluted sample was inoculated in 4 wells; and the waste solution in the 96-well plate was discarded, the 96-well plate was washed with PBS 3 times, the virus solution was inoculated into the 96-well plate at 100 μL/well, the plate was cultured in a 37° C. cell culture incubator for 3 days after being shaken well, cytopathic effects were observed, and the TCID₅₀ was statistically calculated. The results are shown in FIG. 1. The siRNA molecules of different doses in the present invention have different degrees of inhibitory effect on influenza A virus subtypes such as H1N1, H5N1, and H7N9, where M1-1 has the best inhibiting effects and is close to Oseltamivir.

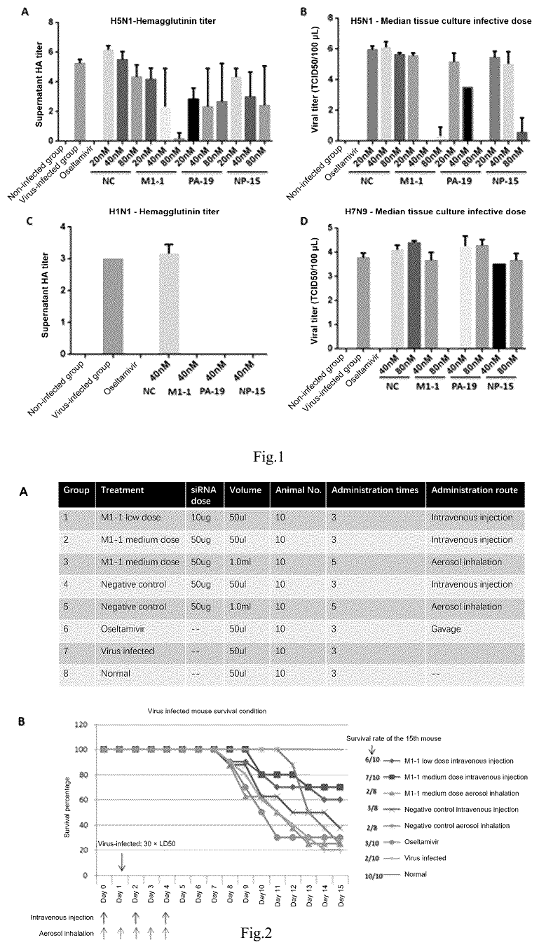

In in-vivo animal experiments in mice, the effectiveness of anti-influenza candidate drugs was evaluated by two ways of intravenous injection and respiratory tract aerosol inhalation administration (FIG. 2A). A highly pathogenic avian influenza H5N1 strain was used as the challenge (30 LD₅₀) to ensure that most or all of the mice in the “virus-infected group” were able to die. The results (FIG. 2B) show that the survival rate of mice with intravenous injection of M1-1 at medium or low doses (70% and 60% respectively) was significantly higher than that (37.5%) of the negative control group (NC) with intravenous injection and that (20%) of the virus-infected group with intravenous injection. It can be seen that the anti-influenza virus siRNA molecule of the present invention can significantly inhibit the infection and replication process of various influenza A viruses in vitro and in vivo.

### Embodiment 2. Screening and verification of siRNA molecules for targeting inhibition of PD-1 and PD-L1 gene expression

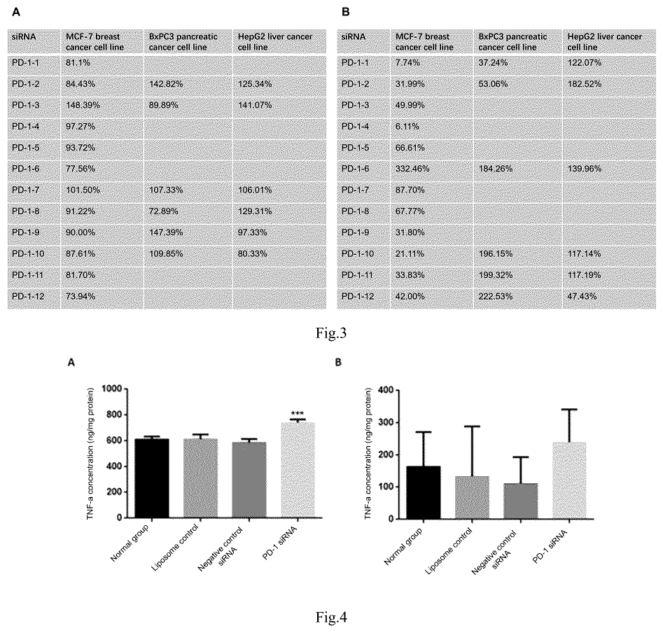

After selecting human-mouse homologous sequences to design the siRNA molecules against PD-1 and PD-L1 genes, we (Suzhou Biosyntech) were commissioned to synthesize these siRNA molecules, and then human tumor cell lines MCF-1, BxPC3, and HepG2 were used to determine the inhibitory effect of the siRNA molecules on PD-1 or PD-L1 expression. sA specific quantity of cells were inoculated into a 6-well plate or a 12-well plate and cultured for more than 6 hours to cause cell adherence. Then, various siRNAs were transfected by Lipofectamine 2000 liposome transfection reagents according to operating instructions, and then cultured for 48 hours after transfection. After total RNAs of cells were extracted (a tissue/cell RNA rapid extraction kit, Beijing Mei5 Biotechnology), the concentration was determined with a micro UV spectrophotometer (MicroDrop, Bio-DEL), and 100-500 ng of total RNAs were taken for reverse transcription (first-strand cDNA reverse transcription kit, Beijing Mei5 Biotechnology). Finally, a fluorescent quantitative PCR amplification kit (Realtime PCR Super mix (SYBRgreen, with anti-Taq), Beijing Mei5 Biotechnology) was used for analysis and detection on a fluorescent PCR instrument (QuantStudio 3, ABI). The results are shown in FIG. 3. The siRNA against PD-1 or PD-L1 can inhibit the expression of corresponding target genes to varying degrees. Accordingly, the most efficient siRNA molecule for each target gene can be selected.

In order to analyze the immunostimulatory effect of the PD-1, a siRNA molecule (PD-1-10) of the PD-1 was selected and transfected into a mouse RAW264.7 macrophage line, and after 28 hours of culture, the concentration of a tumor necrosis factor-α (TNF-α) in the cell culture supernatant and the cell lysate (protein lysate) was determined. The results are shown in FIG. 4. In the culture supernatant of mouse macrophages treated with PD-1, the concentration of TNF-α was significantly increased (FIG. 4A), and the content of TNF-α in the protein lysate was also increased (no statistically significant differences, FIG. 4B). It can be seen that after siRNA is used for inhibiting the PD-1 expression, the function of immune cells can be activated. While anti-influenza virus siRNA was used for inhibiting virus infection in mammals, the siRNA of the PD-1 was used for activating the immune system of the body, so that anti-virus immunity can be effectively strengthened, and the viruses can be more effectively removed.

### Embodiment 3. Inhibition of Influenza Viruses Through Combination of siRNA Molecule and mRNA Vaccine

Many studies have confirmed that siRNA is an efficient anti-virus method, and as confirmed in clinical application, mRNA vaccines can effectively protect human bodies from infection of the new coronavirus (SARS-CoV-2). In the present invention, the anti-influenza virus siRNA molecule is used in combination with influenza mRNA vaccines to efficiently and specifically eliminate pathogens. In the example, as shown in FIG. 5, provided was a mRNA vaccine containing a cap structure, a 5′-untranslated region, an open reading frame (ORF), a 3′-untranslated region, and a polyadenylic acid (PolyA) tail, where the ORF is a gene sequence encoding a specific protein of influenza viruses. The mRNA and an HKP polypeptide nano-introduction system were mixed in a specific ratio to prepare nanoparticles. After the nanoparticles were intramuscularly injected into mammals, the mRNA was translated into proteins and secreted outside the cells to be recognized by antigen presenting cells (APC) and stimulate the immune system of the body, thus forming an antiviral immune protection mechanism.

At the same time or at a specific time after mRNA treatment, an effective dose of the siRNA nano-drug formulation is administered to the mammals. After entering body cells, the antiviral siRNA molecules form a RNA-induced silencing complex (RISC) with a specific enzyme/protein. After the sense strand is dissociated, the antisense strand carries the entire complex and binds to the viral RNA, and the viral RNA was degraded through the RNAi mechanism, so that virus-specific genes are prevented from being expressed into proteins/enzymes, and the viruses cannot complete replication of the life cycle. The mRNA vaccine was used in combination with the siRNA molecules, so that infection and replication of influenza viruses can be efficiently blocked.

### Embodiment 4. Conjugation of Anti-Influenza Virus siRNA Molecules to Anti-Influenza Drugs

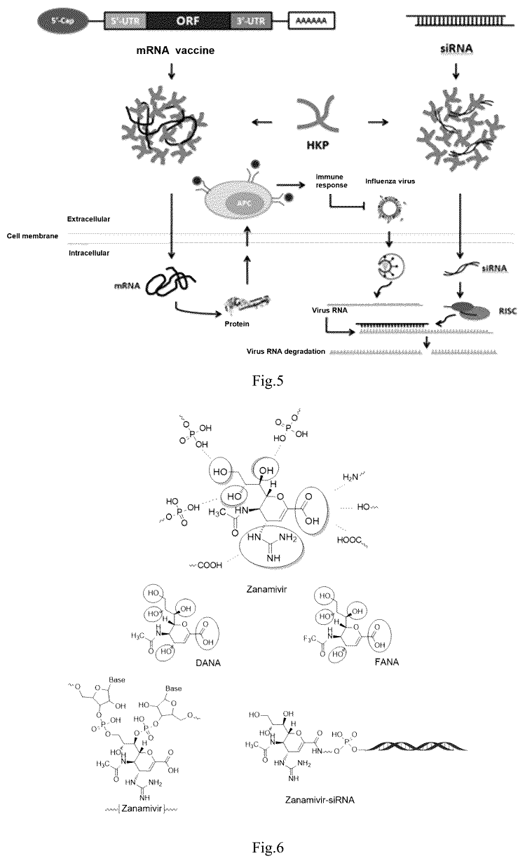

The drug Zanamivir contains hydroxyl groups, carboxyl groups, and guanidine active groups, and Zanamivir is obtained by substituting the hydroxyl group at the C-4 position in DANA with a guanidine group. Among the groups, the guanidine group can be combined with two amino acids Glu119, Glu227 or Asp115 in the S2 region of influenza virus neuraminidase (NA) to improve the enzyme inhibiting activity in vitro. Therefore, the guanidine group plays an important role in the inhibition of NA, and the integrity of the guanidine group should be maintained as much as possible. To this end, the present invention designs two modification methods to combine the drug with antiviral siRNA: one method is to condense phosphate groups and hydroxyl groups into phosphate esters, and block Zanamvir in the siRNA molecules (FIG. 6, - - - [Zanamivir] - - - ); and the other method is to directly link Zanamvir to the end of siRNA or link through the linking compound (Linker). In one aspect of the embodiment, the phosphate group at the 5′ end of siRNA can be directly coupled to the hydroxyl group of Zanamvir; in another aspect of the embodiment, the Linker (such as PEG) is introduced, with one end linked to the carboxyl group of Zanamvir, and the other end to the phosphate group at the 5′ end of the siRNA (FIG. 6, Zanamivir-siRNA) or to the hydroxyl group at the 3′ end. DANA and FANA have similar structural formulas, and can be combined with siRNA by the two methods to screen out binding substances that have the therapeutic effects.

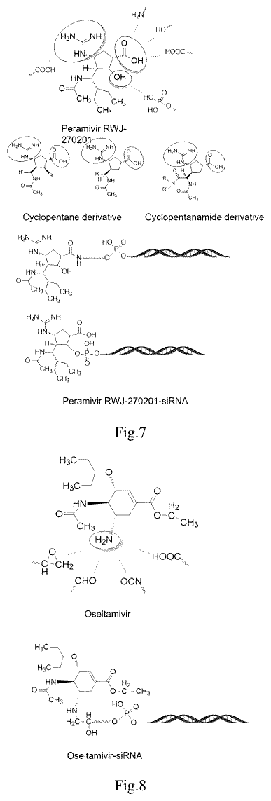

The structural formula of Peramivir (RWJ-270201) contains guanidine groups, hydroxyl groups, and carboxyl active groups. Among them, the hydroxyl group can be condensed with the phosphate group at the 5′ end of the siRNA or the hydroxyl group at the 3′ end of the siRNA, and the retained carboxyl group can be combined with Arg292, Arg371, and Arg118 in the S1 region of the influenza viruses NA. The carboxyl group can be linked to siRNA through Linker. As shown in FIG. 7, for Peramivir RWJ-270201-siRNA, the amino group at one end of the Linker reacts with the carboxyl group to form an amide bond, and the hydroxyl group at the other end of the Linker reacts with the phosphate group to form the phosphate esters. In addition, the Linker may contain responsive groups such as disulfide bonds, which can be cleaved under specific conditions, so that the drug and siRNA can have a synergistic effect to improve the therapeutic effects. While in case of retaining the guanidine group, cyclopentane derivatives and cyclopentanamide derivatives of the drug can couple the siRNA by the above method.

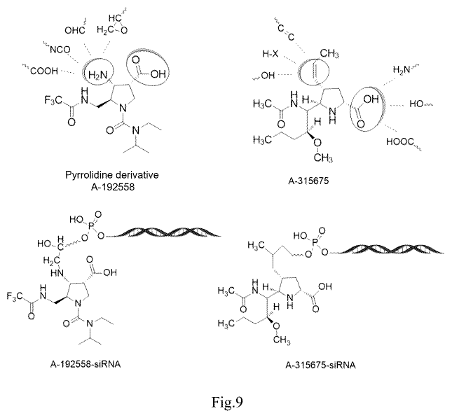

The amino groups of Oseltamivir are the main functional groups and the active groups. The amino groups can react with the epoxy groups, aldehyde groups, isocyanate groups, and carboxyl groups. As shown in FIG. 8, for Oseltamivir-siRNA, the hydroxyl group at one end of the Linker reacts with the phosphate group at the end of the siRNA, and the epoxy group at the other end of the Linker undergoes a nucleophilic reaction with the amino groups of the Oseltamivir. The secondary amine is weaker in alkalinity than the primary amine, but it can still bind to the carboxyl groups of Glu119, Glu227 or Asp115 in the S2 region of influenza virus NA proteins.

In the structure of A-192558, the modifiable active groups are amino groups and carboxyl groups. If the amino groups are used for modification, the epoxy group at one end of the Linker reacts with the amino groups, and the hydroxyl group at the other end of the Linker reacts with the phosphate group of siRNA (as shown in FIG. 9, A-192558-siRNA). The retained carboxyl group can be combined with the guanidine groups of Arg292, Arg371 and Arg118 in the S1 region of NA. If the carboxyl group is used for coupling, the retained amino groups are combined with the carboxyl group of Glu119, Glu227 or Asp115 in the S2 region of NA. In the structure of another anti-influenza small molecule drug A-315675, the modifiable groups include carbon-carbon double bonds and carboxyl groups, and the carbon-carbon double bonds can undergo addition reactions with water, halogen and the like, or the modifiable groups can also undergo polymerization reactions with the carbon-carbon double bonds, carbon-carbon triple bonds, and other olefins and alkynes. As shown in FIG. 9, for the A-315675-siRNA, the conjugation is realized by an addition reaction with the carbon-carbon double bonds through hydrogen halide and then linkage to the Linker through a substitution reaction.

### Embodiment 5. Anti-Influenza Virus siRNA Molecule and Polypeptide Nano-Drug Formulation of mRNA Vaccine

In the present invention, a polymer, especially a histidine-lysine polymer (HKP) is used for encapsulating nucleic acid drug molecules, including siRNA and mRNA, to prepare nano-drug particles. In one aspect of the embodiment, nanoparticles are prepared from the HKP and siRNA molecules, where the nanoparticles are about 30 nm to about 300 nm in diameter. The HKP is H3K(+H)4b, and includes the structure (R)K(R))-K(R)-(R)K(X), where R=KHHHKHHHKHHHHKHHHK, K=lysine, and H=Histidine. The HKP and the siRNA molecules self-assemble into nanoparticles or made into the nanoparticles. In another aspect of the embodiment, the nanoparticles are prepared from the HKP and mRNA vaccine molecules, where the nanoparticles are about 30 nm to about 400 nm in diameter. The HKP is H3K(+H)4b, and the HKP and mRNA molecules can self-assemble into nanoparticles or be made into the nanoparticles. In another aspect of the embodiment, the HKP can be used for encapsulating the siRNA and the mRNA molecules at the same time to form the nanoparticles which are 30 nm-400 nm or even larger in diameter.

In the present invention, after HKP polypeptide is used to encapsulate siRNA or/and mRNA to form the nanoparticles, a series of determination methods are established to characterize the physicochemical properties of the nano-drug formulations, including particle size, surface potential, morphological studies, loading efficiency of mRNA or siRNA, biological activity, etc. In one aspect of the embodiment, a Zetasizer Nano ZS (Malvern Instruments, UK) was used to determine the size and potential of the nano-drug formulation particles. In another aspect of the embodiment, a real-time quantitative fluorescence PCR method was used to determine the inhibitory effect of the siRNA on the expression of viral target genes. In another aspect of the embodiment, after the cells were treated with mRNA nano-drugs, the expressed proteins or polypeptides were identified and quantified by RPHPLC using an analytical column C18 (2S0 mm×2.1 mm; Phenomenex).

### Embodiment 6. Aerosol Inhalation Administration for Preventing and Treating Respiratory Virus Infection Diseases

In order to test the development potential of the nano-drug formulation prepared from HKP polypeptides and siRNA in the treatment of respiratory tract/lung diseases by aerosol inhalation administration, the siRNA (unlabeled or fluorescently-labeled) targeting specific target genes was delivered into the respiratory system through the mouth and nose by aerosol inhalation. The mice were placed in a closed chamber, and the nano-drug formulation was placed in an atomizing cup. After a spray port of an atomizer was airtightly connected to a cavity, a power supply was turned on for atomization for a certain period of time, and then the situation of the siRNA entering the lung and the efficiency of the siRNA for inhibiting the expression of target genes were determined.

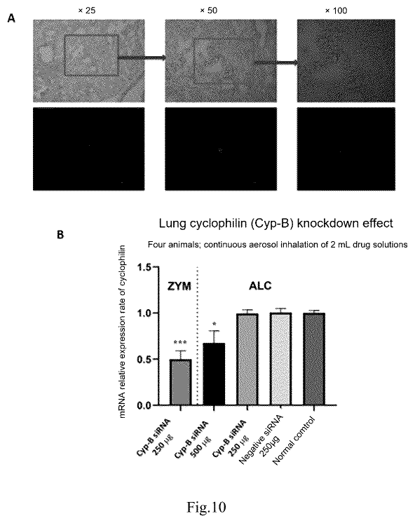

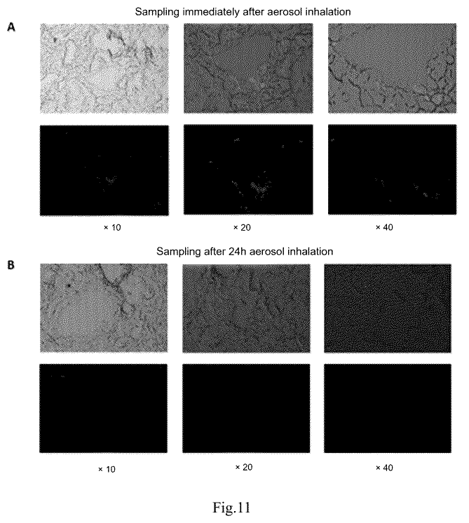

The fluorescently-labeled siRNA (AF647-siRNA, Qiagen) or the siRNA against Cyclophilin-B (Suzhou Biosyntech) was atomized through an ultrasonic atomizer (ALC) or a hand-held atomizer (ZYM), and the efficiency of the siRNA entering the lungs of the mice was determined. In one aspect of the embodiment, the drugs were administrated in the aerosol inhalation manner with a short duration and a short interval and given in the atomizing chamber at one time (2 mL), and the atomization is performed: first, an atomizing chamber was filled for about 30-40 seconds and then stopped, after 20 seconds, atomization was on for 10 seconds and then off for 20 seconds, and atomization was on for 10 seconds and then off for 20 seconds. The atomization and stopping processes were cycled until the atomization was completed. After aerosol inhalation administration, some mice were sacrificed, the lungs were separated, and the siRNA fluorescence was determined. 24 hours after the aerosol inhalation administration, some mice were sacrificed, the lungs were separated, tissue RNAs were extracted, and the expression of Cyclophilin-B genes was determined by PCR. As shown in FIG. 10, the drugs can effectively reach the lungs (enriched in the alveoli), and have a significant inhibitory effect on the target genes.

In another aspect of the embodiment, the drugs were administrated in the aerosol inhalation manner with a short duration and a short interval and given in the atomizing chamber at one time (2 mL, complete atomizaton): first, the atomizing chamber was filled for about 1 minute and then stopped, after 1 minute, atomization was on for 1 minute and then off for 1 minute, and atomization was on for 1 minute and then off for 1 minute. The process was cycled until all the drug solutions were atomized. When the aerosol inhalation administration was completed or 24 hours after administration, some mice were sacrificed, the lungs were separated, and the siRNA fluorescence was determined. As shown in FIG. 11, whether detection was performed immediately after the aerosol inhalation administration (FIG. 11A) or 24 hours after the administration (FIG. 11B), the siRNAs can be detected in the lungs, indicating that after entering the lungs, the siRNAs can be retained in the lungs for a certain period of time to ensure full effectiveness.

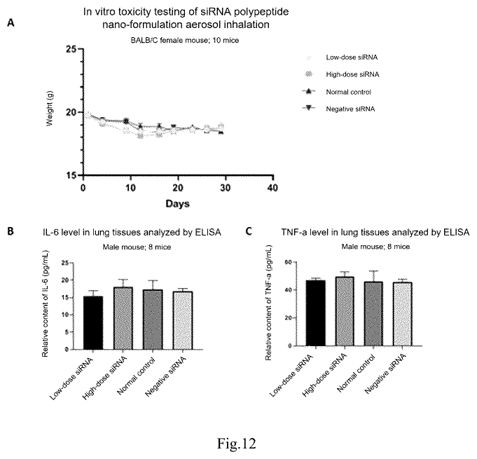

In another aspect of the example, the adverse effects on the body after aerosol inhalation administration (hand-held atomizer ZYM) of the siRNAs were determined. The results show that aerosol inhalation administration has no significant effects on the body weight of mice (FIG. 12A); and at different times after aerosol inhalation administration, the inflammatory factors in the lungs were determined, and the results indicate that neither IL-6 nor TNF-α has significant changes (FIGS. 12B-C). It can be seen that the siRNAs have no significant toxic and side effects after aerosol inhalation, and can be used for treating various respiratory and pulmonary diseases, especially respiratory virus infection diseases such as influenza A virus infection.

The above-mentioned embodiments are only intended to illustrate the technical concept and characteristics of the present invention, and the purpose thereof is to enable those skilled in the technology to understand the content of the present invention and implement them accordingly, but they cannot limit the protection scope of the present invention. All equivalent changes or modifications made actually according to the spirit of the present invention should be included within the protection scope of the present invention.

### SUMMARY OF THE INVENTION

siRNA Molecules Against Influenza a Virus Infection

Provided is a small interfering nucleotide (siRNA) molecule that can effectively inhibit influenza A viruses, where the molecule is selected from the new siRNA molecules in Table 1. The siRNA molecule for inhibiting influenza A viruses is designed for the conserved gene sequences of various subtypes of influenza A virus, including but not limited to G4 EA H1N1 virus strains (A/swine/Hebei/0116/2017 (H1N1) and A/swine/Jiangsu/J004/2018 (H1N1), etc.), H1N1 virus strains (A/PuertoRico/8/1934 and A/California/07/2009, etc.), H5N1 virus strains (A/Vietnam/1194/2004 etc.), H7N9 virus strains (A/Shanghai/CN02/2013 etc.) and H3N2 virus strains (A/Texas/50/2012 etc.) etc. with “all characteristics” highly adapted to infection of humans. The siRNA molecule is designed against the homologous sequences of the above strains. The length of the siRNA molecule is 19-30 base pairs, and preferably, the length of the siRNA molecule is 21 or 25 base pairs. The GC content of the siRNA molecule is 30%-70%, and preferably, the GC content of the siRNA molecule is 40%-60%.

TABLE 1
siRNA molecules for broad-spectrum inhibition of influenza A viruses

| Name of | | | SEQ ID | | SEQ ID |
|---|---|---|---|---|---|
| siRNA | Length | sense strand (5′-3′) | No.. | antisense strand (5′-3′) | No.. |
| PB2-1 | 19 + dTdT | GUGGACCAUAUGGCCAUAAdTdT | 1 | UUAUGGCCAUAUGGUCCACdTdT | 98 |
| PB2-2 | 19 + dTdT | CGGGACUCUAGCAUACUUAdTdT | 2 | UAAGUAUGCUAGAGUCCCGdTdT | 99 |
| PB2-3 | 19 + dTdT | GACUCUAGCAUACUUACUGdTdT | 3 | CAGUAAGUAUGCUAGAGUCdTdT | 100 |
| PB2-4 | 19 + dTdT | CUCUAGCAUACUUACUGACdTdT | 4 | GUCAGUAAGUAUGCUAGAGdTdT | 101 |
| PB2-5 | 19 + dTdT | CUAGCAUACUUACUGACAGdTdT | 5 | CUGUCAGUAAGUAUGCUAGdTdT | 102 |
| PB2-6 | 19 + dTdT | GCAUACUUACUGACAGCCAdTdT | 6 | UGGCUGUCAGUAAGUAUGCdTdT | 103 |
| PB2-7 | 19 + dTdT | CUUACUGACAGCCAGACAGdTdT | 7 | CUGUCUGGCUGUCAGUAAGdTdT | 104 |
| PB2-8 | 19 + dTdT | GCCAGACAGCGACCAAAAGdTdT | 8 | CUUUUGGUCGCUGUCUGGCdTdT | 105 |
| PB2-9 | 19 + dTdT | GCGACCAAAAGAAUUCGGAdTdT | 9 | UCCGAAUUCUUUUGGUCGCdTdT | 106 |
| PB2-10 | 19 + dTdT | GAAUUCGGAUGGCCAUCAAdTdT | 10 | UUGAUGGCCAUCCGAAUUCdTdT | 107 |
| PB2-11 | 25 | GAAACGAAAACGGGACUCUAGCAUA | 11 | UAUGCUAGAGUCCCGUUUUCGUUUC | 108 |
| PB2-12 | 25 | CGAAAACGGGACUCUAGCAUACUUA | 12 | UAAGUAUGCUAGAGUCCCGUUUUCG | 109 |
| PB2-13 | 25 | GCAUACUUACUGACAGCCAGACAGC | 13 | GCUGUCUGGCUGUCAGUAAGUAUGC | 110 |
| PB2-14 | 25 | CUUACUGACAGCCAGACAGCGACCA | 14 | UGGUCGCUGUCUGGCUGUCAGUAAG | 111 |
| PB2-15 | 25 | GACAGCCAGACAGCGACCAAAAGAA | 15 | UUCUUUUGGUCGCUGUCUGGCUGUC | 112 |
| PB2-16 | 25 | CCAAAAGAAUUCGGAUGGCCAUCAA | 16 | UUGAUGGCCAUCCGAAUUCUUUUGG | 113 |
| PB2-17 | 25 | AUGGCCAUCCGAAUUCUUUUGGUCG | 17 | CGACCAAAAGAAUUCGGAUGGCCAU | 114 |
| PB2-18 | 25 | UUGAUGGCCAUCCGAAUUCUUUUGG | 18 | CCAAAAGAAUUCGGAUGGCCAUCAA | 115 |
| PB2-19 | 19 + dTdT | GGGAACAGAUGUACACUCCdTdT | 19 | GGAGUGUACAUCUGUUCCCdTdT | 116 |
| PB1-1 | 19 + dTdT | CACAUUCCCUUAUACUGGAdTdT | 20 | UCCAGUAUAAGGGAAUGUGdTdT | 117 |
| PB1-2 | 19 + dTdT | CCUCCAUACAGCCAUGGAAdTdT | 21 | UUCCAUGGCUGUAUGGAGGdTdT | 118 |
| PB1-3 | 19 + dTdT | CAGCCAUGGAACAGGAACAdTdT | 22 | UGUUCCUGUUCCAUGGCUGdTdT | 119 |
| PB1-4 | 19 + dTdT | CCAUGGAACAGGAACAGGAdTdT | 23 | UCCUGUUCCUGUUCCAUGGdTdT | 120 |
| PB1-5 | 19 + dTdT | GAACAGGAACAGGAUACACdTdT | 24 | GUGUAUCCUGUUCCUGUUCdTdT | 121 |
| PB1-6 | 19 + dTdT | GAUGAUGGGCAUGUUCAACdTdT | 25 | GUUGAACAUGCCCAUCAUCdTdT | 122 |
| PB1-7 | 19 + dTdT | GUGGAGGCCAUGGUGUCUAdTdT | 26 | UAGACACCAUGGCCUCCACdTdT | 123 |
| PB1-8 | 19 + dTdT | GAGAUCAUGAAGAUCUGUUdTdT | 27 | AACAGAUCUUCAUGAUCUCdTdT | 124 |
| PB1-9 | 19 + dTdT | GAUCAUGAAGAUCUGUUCCdTdT | 28 | GGAACAGAUCUUCAUGAUCdTdT | 125 |
| PB1-10 | 19 + dTdT | CAUGAAGAUCUGUUCCACCdTdT | 29 | GGUGGAACAGAUCUUCAUGdTdT | 126 |
| PB1-11 | 19 + dTdT | GAAGAUCUGUUCCACCAUUdTdT | 30 | AAUGGUGGAACAGAUCUUCdTdT | 127 |
| PB1-12 | 25 | CUUAUACUGGAGAUCCUCCAUACAG | 31 | CUGUAUGGAGGAUCUCCAGUAUAAG | 128 |
| PB1-13 | 25 | CUGGAGAUCCUCCAUACAGCCAUGG | 32 | CCAUGGCUGUAUGGAGGAUCUCCAG | 129 |
| PB1-14 | 25 | GCCAUGGAACAGGAACAGGAUACAC | 33 | GUGUAUCCUGUUCCUGUUCCAUGGC | 130 |
| PB1-15 | 25 | CAUGGUGGAGGCCAUGGUGUCUAGG | 34 | CCUAGACACCAUGGCCUCCACCAUG | 131 |
| PB1-16 | 25 | GAUCAUGAAGAUCUGUUCCACCAUU | 35 | AAUGGUGGAACAGAUCUUCAUGAUC | 132 |
| PB1-17 | 25 | CAUGAAGAUCUGUUCCACCAUUGAA | 36 | UUCAAUGGUGGAACAGAUCUUCAUG | 133 |
| PA-1 | 19 + dTdT | GGGAUUCCUUUCGUCAGUCdTdT | 37 | GACUGACGAAAGGAAUCCCdTdT | 134 |
| PA-2 | 19 + dTdT | GAUUCCUUUCGUCAGUCCGdTdT | 38 | CGGACUGACGAAAGGAAUCdTdT | 135 |
| PA-3 | 19 + dTdT | GCCUGAUUAAUGAUCCCUGdTdT | 39 | CAGGGAUCAUUAAUCAGGCdTdT | 136 |
| PA-4 | 19 + dTdT | CUGAUUAAUGAUCCCUGGGdTdT | 40 | CCCAGGGAUCAUUAAUCAGdTdT | 137 |
| PA-5 | 19 + dTdT | GAUUAAUGAUCCCUGGGUUdTdT | 41 | AACCCAGGGAUCAUUAAUCdTdT | 138 |
| PA-6 | 19 + dTdT | GAUCCCUGGGUUUUGCUUAdTdT | 42 | UAAGCAAAACCCAGGGAUCdTdT | 139 |
| PA-7 | 19 + dTdT | CUGGGUUUUGCUUAAUGCAdTdT | 43 | UGCAUUAAGCAAAACCCAGdTdT | 140 |
| PA-8 | 19 + dTdT | CUUAAUGCAUCUUGGUUCAdTdT | 44 | UGAACCAAGAUGCAUUAAGdTdT | 141 |
| PA-9 | 19 + dTdT | GCAUCUUGGUUCAACUCCUdTdT | 45 | AGGAGUUGAACCAAGAUGCdTdT | 142 |
| PA-10 | 19 + dTdT | CUUGGUUCAACUCCUUCCUdTdT | 46 | AGGAAGGAGUUGAACCAAGdTdT | 143 |
| PA-11 | 19 + dTdT | GUUCAACUCCUUCCUCACAdTdT | 47 | UGUGAGGAAGGAGUUGAACdTdT | 144 |
| PA-12 | 19 + dTdT | CUCCUUCCUCACACAUGCAdTdT | 48 | UGCAUGUGUGAGGAAGGAGdTdT | 145 |
| PA-13 | 25 | GAAAGAAUAUGGGGAAGAUCCGAAA | 49 | UUUCGGAUCUUCCCCAUAUUCUUUC | 146 |
| PA-14 | 25 | GCCUGAUUAAUGAUCCCUGGGUUUU | 50 | AAAACCCAGGGAUCAUUAAUCAGGC | 147 |
| PA-15 | 25 | CUUAAUGCAUCUUGGUUCAACUCCU | 51 | AGGAGUUGAACCAAGAUGCAUUAAG | 148 |
| PA-16 | 25 | GCAUCUUGGUUCAACUCCUUCCUCA | 52 | UGAGGAAGGAGUUGAACCAAGAUGC | 149 |
| PA-17 | 25 | CUUGGUUCAACUCCUUCCUCACACA | 53 | UGUGUGAGGAAGGAGUUGAACCAAG | 150 |
| PA-18 | 25 | GUUCAACUCCUUCCUCACACAUGCA | 54 | UGCAUGUGUGAGGAAGGAGUUGAAC | 151 |
| PA-19 | 19 + dTdT | GCAAUUGAGGAGUGCCUGAdTdT | 55 | UCAGGCACUCCUCAAUUGCdTdT | 152 |
| NP-1 | 19 + dTdT | GAGUCUUCGAGCUCUCGGAdTdT | 56 | UCCGAGAGCUCGAAGACUCdTdT | 153 |
| NP-2 | 19 + dTdT | CUUCGAGCUCUCGGACGAAdTdT | 57 | UUCGUCCGAGAGCUCGAAGdTdT | 154 |
| NP-3 | 19 + dTdT | CGAGCUCUCGGACGAAAAGdTdT | 58 | CUUUUCGUCCGAGAGCUCGdTdT | 155 |
| NP-4 | 19 + dTdT | GCUCUCGGACGAAAAGGCAdTdT | 59 | UGCCUUUUCGUCCGAGAGCdTdT | 156 |
| NP-5 | 19 + dTdT | CUCGGACGAAAAGGCAACGdTdT | 60 | CGUUGCCUUUUCGUCCGAGdTdT | 157 |
| NP-6 | 19 + dTdT | CGGACGAAAAGGCAACGAAdTdT | 61 | UUCGUUGCCUUUUCGUCCGdTdT | 158 |
| NP-7 | 19 + dTdT | CGAAAAGGCAACGAACCCGdTdT | 62 | CGGGUUCGUUGCCUUUUCGdTdT | 159 |
| NP-8 | 19 + dTdT | GAAAAGGCAACGAACCCGAdTdT | 63 | UCGGGUUCGUUGCCUUUUCdTdT | 160 |
| NP-9 | 19 + dTdT | CCUUUGACAUGAGUAAUGAdTdT | 64 | UCAUUACUCAUGUCAAAGGdTdT | 161 |
| NP-10 | 19 + dTdT | CUUAUUUCUUCGGAGACAAdTdT | 65 | UUGUCUCCGAAGAAAUAAGdTdT | 162 |
| NP-11 | 25 | GAGUCUUCGAGCUCUCGGACGAAAA | 66 | UUUUCGUCCGAGAGCUCGAAGACUC | 163 |
| NP-12 | 25 | CGAAAAGGCAACGAACCCGAUCGUG | 67 | CACGAUCGGGUUCGUUGCCUUUUCG | 164 |
| NP-13 | 25 | GAAAAGGCAACGAACCCGAUCGUGC | 68 | GCACGAUCGGGUUCGUUGCCUUUUC | 165 |
| NP-14 | 25 | CUUAUUUCUUCGGAGACAAUGCAGA | 69 | UCUGCAUUGUCUCCGAAGAAAUAAG | 166 |
| NP-15 | 19 + dTdT | CUCCGAAGAAAUAAGAUCCdTdT | 70 | GGAUCUUAUUUCUUCGGAGdTdT | 167 |
| NA-1 | 19 + dTdT | GUCUUGGCCAGACGGUGCUdTdT | 71 | AGCACCGUCUGGCCAAGACdTdT | 168 |
| NA-2 | 19 + dTdT | GGCCAGACGGUGCUGAGUUdTdT | 72 | AACUCAGCACCGUCUGGCCdTdT | 169 |
| NA-3 | 19 + dTdT | GACGGUGCUGAGUUGCCAUdTdT | 73 | AUGGCAACUCAGCACCGUCdTdT | 170 |
| NA-4 | 25 | GGCCAGACGGUGCUGAGUUGCCAUU | 74 | AAUGGCAACUCAGCACCGUCUGGCC | 171 |
| M-1 | 19 + dTdT | GUCUUCUAACCGAGGUCGAdTdT | 75 | UCGACCUCGGUUAGAAGACdTdT | 172 |
| M-2 | 19 + dTdT | CUUCUAACCGAGGUCGAAAdTdT | 76 | UUUCGACCUCGGUUAGAAGdTdT | 173 |
| M-3 | 19 + dTdT | CUAACCGAGGUCGAAACGUdTdT | 77 | ACGUUUCGACCUCGGUUAGdTdT | 174 |
| M-4 | 19 + dTdT | CCGAGGUCGAAACGUACGUdTdT | 78 | ACGUACGUUUCGACCUCGGdTdT | 175 |
| M-5 | 19 + dTdT | CGAGGUCGAAACGUACGUUdTdT | 79 | AACGUACGUUUCGACCUCGdTdT | 176 |
| M-6 | 19 + dTdT | CCCUCAAAGCCGAGAUCGCdTdT | 80 | GCGAUCUCGGCUUUGAGGGdTdT | 177 |
| M-7 | 19 + dTdT | GGCUAAAGACAAGACCAAUdTdT | 81 | AUUGGUCUUGUCUUUAGCCdTdT | 178 |
| M-8 | 19 + dTdT | CACGCUCACCGUGCCCAGUdTdT | 82 | ACUGGGCACGGUGAGCGUGdTdT | 179 |
| M-9 | 19 + dTdT | CGCUCACCGUGCCCAGUGAdTdT | 83 | UCACUGGGCACGGUGAGCGdTdT | 180 |
| M-10 | 19 + dTdT | CGUGCCCAGUGAGCGAGGAdTdT | 84 | UCCUCGCUCACUGGGCACGdTdT | 181 |
| M-11 | 19 + dTdT | CCAGUGAGCGAGGACUGCAdTdT | 85 | UGCAGUCCUCGCUCACUGGdTdT | 182 |
| M-12 | 19 + dTdT | GAGCGAGGACUGCAGCGUAdTdT | 86 | UACGCUGCAGUCCUCGCUCdTdT | 183 |
| M-13 | 19 + dTdT | GGACUGCAGCGUAGACGCUdTdT | 87 | AGCGUCUACGCUGCAGUCCdTdT | 184 |
| M-14 | 25 | GUCUUCUAACCGAGGUCGAAACGUA | 88 | UACGUUUCGACCUCGGUUAGAAGAC | 185 |
| M-15 | 25 | CACGCUCACCGUGCCCAGUGAGCGA | 89 | UCGCUCACUGGGCACGGUGAGCGUG | 186 |
| M-16 | 25 | GCUCACCGUGCCCAGUGAGCGAGGA | 90 | UCCUCGCUCACUGGGCACGGUGAGC | 187 |
| M-17 | 25 | CCGUGCCCAGUGAGCGAGGACUGCA | 91 | UGCAGUCCUCGCUCACUGGGCACGG | 188 |
| M-18 | 25 | CCCAGUGAGCGAGGACUGCAGCGUA | 92 | UACGCUGCAGUCCUCGCUCACUGGG | 189 |
| M-19 | 25 | CAGUGAGCGAGGACUGCAGCGUAGA | 93 | UCUACGCUGCAGUCCUCGCUCACUG | 190 |
| M-20 | 25 | GACUGCAGCGUAGACGCUUUGUCCA | 94 | UGGACAAAGCGUCUACGCUGCAGUC | 191 |
| M-21 | 19 + dTdT | UUCGACCUCGGUUAGAAGAdTdT | 95 | UCUUCUAACCGAGGUCGAAdTdT | 192 |
| M1-1 | 25 | UACGCUGCAGUCCUCGCUCACUGGG | 96 | CCCAGUGAGCGAGGACUGCAGCGUA | 193 |
| M2-1 | 19 + dTdT | AUCCACAGCAUUCUGCUGUdTdT | 97 | ACAGCAGAAUGCUGUGGAUdTdT | 194 |

Combination with siRNA Molecules of PD-L1

A PD-1/PD-L1 signaling pathway is important for antiviral immune effects and can affect the severity of immune pathological damage caused by pathogen infection in bodies. In chronic viral infection, a programmed death factor 1 (PD1) is highly expressed on the surface of CD8+ T cells, which is one of the markers of CD8+ T cell exhaustion. In recent years. through studies, it has been found that regulatory T cells also highly express inhibitory molecules such as PD1 in chronic viral infection, which may be related to increased viral load or increased inhibition effect of antiviral T cell responses. During the acute phase of viral infection, virus-specific T cells rapidly up-regulate the co-inhibitory receptor PD-1 after the antigen was recognized, and directly up-regulate PD-L1 on hematopoietic and non-hematopoietic cells through PRR signaling or indirectly up-regulate PD-L1 by inducing the release of IFN and other inflammatory cytokines. Viruses can also control the balance of the immune system, thereby preventing an effective antiviral immune response to help the persistence of pathogens in an organism. After blocking PD1/PD-L1 signaling pathways on surfaces of the regulatory T cells and depleted CD8+ T cells, the function of the depleted CD8+ T cells can be reversed, which brings a new opportunity for targeted therapeutic strategies for treating chronic viral infection diseases.

One study showed that respiratory syncytial virus (RSV) induced PD-L1 expression on bronchial epithelial cells, thereby inhibiting the antiviral effects of local CD8+ T cells^{[25]}, indicating that the interaction between epithelial cells and T cells during viral infection would be affected, which is beneficial to viral infection and replication. Previous studies have also shown that during the acute phase of infection, hepatitis B virus infection significantly increases PD-1 expression on effector T cells before becoming persistent or latent chronic infection^{[28]}. In a recent study, the level of PD-L1 was significantly up-regulated in H9N2 virus-infected rat pulmonary microvascular endothelial cells (RPMEC), and viral infection-induced PD-L1 expression transmitted the negative signal to the migrating T cells, resulting in down-regulation of antiviral cytokines and reduced production of cytotoxic proteins^{[29]}.

In addition to the siRNA molecule against influenza A virus infection, the composition of the present invention also includes a siRNA molecule for specifically inhibiting PD-1 or PD-L1 gene expression in specific cells of the host, and the siRNA molecule is selected from the sequences in Table 2 and Table 3. The siRNA molecule for specifically inhibiting PD-1 or PD-L1 gene expression can inhibit the PD-1 or PD-L1 gene expression in specific cells after reaching a specific part of the body, so that the function of virus-specific T cells can be enhanced, and the siRNA molecule generates synergistic effects with the siRNA molecule against influenza A virus infection to effectively inhibit the virus infection and thoroughly eliminate the viruses in the body. Preferably, the siRNA molecule for inhibiting PD-1 or PD-L1 gene expression has the function of inhibiting expression of the human and mouse PD-1 or PD-L1 genes.

TABLE 2
siRNA molecule for inhibiting PD-1 expression

| | | SEQ | | SEQ |
|---|---|---|---|---|
| No. | sense strand (5′-3′) | ID No. | antisense strand (5′-3′) | ID No. |
| PD-1-1 | ACCCUGCUCAGCCUGCACAAGGUGG | 195 | CCACCUUGUGCAGGCUGAGCAGGGU | 207 |
| PD-1-2 | CCCUGCUCAGCCUGCACAAGGUGGA | 196 | UCCACCUUGUGCAGGCUGAGCAGGG | 208 |
| PD-1-3 | CUGUCCUGCAGCUCCUCCCUCUGCC | 197 | GGCAGAGGGAGGAGCUGCAGGACAG | 209 |
| PD-1-4 | UGUCCUGCAGCUCCUCCCUCUGCCA | 198 | UGGCAGAGGGAGGAGCUGCAGGACA | 210 |
| PD-1-5 | UCCUGCAGCUCCUCCCUCUGCCAGC | 199 | GCUGGCAGAGGGAGGAGCUGCAGGA | 211 |
| PD-1-6 | CCUGCAGCUCCUCCCUCUGCCAGCG | 200 | CGCUGGCAGAGGGAGGAGCUGCAGG | 212 |
| PD-1-7 | CUGCAGCUCCUCCCUCUGCCAGCGC | 201 | GCGCUGGCAGAGGGAGGAGCUGCAG | 213 |
| PD-1-8 | UGCAGCUCCUCCCUCUGCCAGCGCU | 202 | AGCGCUGGCAGAGGGAGGAGCUGCA | 214 |
| PD-1-9 | GCAGCUCCUCCCUCUGCCAGCGCUG | 203 | CAGCGCUGGCAGAGGGAGGAGCUGC | 215 |
| PD-1-10 | CAGCUCCUCCCUCUGCCAGCGCUGU | 204 | ACAGCGCUGGCAGAGGGAGGAGCUG | 216 |
| PD-1-11 | AGCUCCUCCCUCUGCCAGCGCUGUG | 205 | CACAGCGCUGGCAGAGGGAGGAGCU | 217 |
| PD-1-12 | UCCUCCCUCUGCCAGCGCUGUGACA | 206 | UGUCACAGCGCUGGCAGAGGGAGGA | 218 |

TABLE 3
siRNA molecule for inhibiting PD-L1 expression

| | | SEQ | | SEQ |
|---|---|---|---|---|
| No. | sense strand (5′-3′) | ID No. | antisense strand (5′-3′) | ID No. |
| PD-L1-1 | CCAAGGACCUAUAUGUGGUAGAGUA | 219 | UACUCUACCACAUAUAGGUCCUUGG | 231 |
| PD-L1-2 | CGACUACAAGCGAAUUACUGUGAAA | 220 | UUUCACAGUAAUUCGCUUGUAGUCG | 232 |
| PD-L1-3 | CCUCUGAACAUGAACUGACAUGUCA | 221 | UGACAUGUCAGUUCAUGUUCAGAGG | 233 |
| PD-L1-4 | CCAAAUGAAAGGACUCACUUGGUAA | 222 | UUACCAAGUGAGUCCUUUCAUUUGG | 234 |
| PD-L1-5 | GCACUGACAUUCAUCUUCCGUUUAA | 223 | UUAAACGGAAGAUGAAUGUCAGUGC | 235 |
| PD-L1-6 | GGGAUCCAUCCUGUUGUUCCUCAUU | 224 | AAUGAGGAACAACAGGAUGGAUCCC | 236 |
| PD-L1-7 | UCCAUGAAGUGUCAUGAAUCUUGUU | 225 | AACAAGAUUCAUGACACUUCAUGGA | 237 |
| PD-L1-8 | CCCUCUGAUCGUCGAUUGGCAGCUU | 226 | AAGCUGCCAAUCGACGAUCAGAGGG | 238 |
| PD-L1-9 | AAGGAAGAUGAGCAAGUGAUUCAGU | 227 | ACUGAAUCACUUGCUCAUCUUCCUU | 239 |
| PD-L1-10 | CAGCUAACCUCUGUUAUCCUCACUU | 228 | AAGUGAGGAUAACAGAGGUUAGCUG | 240 |
| PD-L1-11 | CAGGCUCUUGCUGUCUGACUCAAAU | 229 | AUUUGAGUCAGACAGCAAGAGCCUG | 241 |
| PD-L1-12 | GACGCAGGCGUUUACUGCUGCAUAA | 230 | UUAUGCAGCAGUAAACGCCUGCGUC | 242 |

Combination with Anti-Influenza Virus Small Molecule Compound

There are two existing strategies against influenza viruses: vaccines and small molecule anti-influenza drugs. Influenza vaccination is the most effective method to prevent influenza. Now there are also trivalent inactivated vaccines and live attenuated vaccines on the market, but the vaccines need to be reconfigured every year to deal with antigenic variation, and the vaccines are long in development cycle and high in cost. These shortcomings make small molecule drugs become the main means of preventing and treating the influenza. Antiviral small molecule compounds currently on the market or in clinical stage mainly include specific influenza virus inhibitors (M2 ion channel blockers, NA inhibitors, PA inhibitors, and PB2 inhibitors) and some broad-spectrum antiviral drugs (Ribavirin, Nitazoxanide, Arbidol hydrochloride, Favipiravir, etc.). However, the rapid emergence of virus resistance to these drugs, bioavailability limitations, adverse reactions and other problems limit the widespread use of these drugs, and at the same time, combination therapy has become a major development direction for the treatment of influenza virus infection. Clinical studies on the combination medication of the various small molecule drugs have been carried out continuously. However, the combined use of various small molecule compounds cannot achieve a very significant synergistic effect due to the similarities in the structure, mechanism of action, bioavailability, and half-life of the drugs, and it is necessary to use drugs with significantly different structures and mechanisms, and significant differences in physical and chemical properties for combination. Only this new composition can significantly inhibit the life cycle of the viruses from multiple perspectives and levels, forming a huge synergistic effect.

In the present invention, the siRNA molecule against influenza A virus infection and the anti-influenza A small molecule compound are combined to exert a synergistic antiviral effect through different mechanisms of action. Preferably, in the composition including the siRNA molecule against influenza A virus infection and the anti-influenza A small molecule compound, the siRNA molecule and the small molecule compound target and inhibit viral internal proteins and viral external proteins respectively. The siRNA molecule against influenza A virus infection targets the expression of viral internal proteins such as PA, PB1, PB2 or NP genes, and the small molecule compound is Oseltamivir, Arbidol, Amantadine or the other molecule that inhibits viral external proteins such as NA, HA, and M proteins. The siRNA molecule against influenza A virus infection targets the gene expression of external proteins such as NA, HA, and M proteins, and the small molecule compound is Favipiravir or Naproxen or the other molecule that inhibits internal proteins such as PA, PB1, PB2 or NP.

In one embodiment, PB2-11 siRNA (sense strand is 5′-GAAACGAAAACGGGACUCUAGCAUA-3′) combined with the NA inhibitor Oseltamivir to simultaneously inhibit viral polymerase genes and neuraminidase.

In another embodiment, the siRNA molecule for inhibiting the M1 proteins (sense strand is 5′-UACGCUGCAGUCCUCGCUCACUGGG-3′) was combined with the influenza virus polymerase inhibitor Favipiravir and the expression or function of two different genes/proteins was simultaneously inhibited.

In another embodiment, NA-1 (sense strand is 5′-GUCUUGGCCAGACGGUGCUdTdT-3′) siRNA, a siRNA molecule for inhibiting polymerase PA genes (sense strand is 5′-GCAAUUGAGGAGUGCCUGAdTdT- -3′) and Ribavirin are combined.

Combination with Influenza mRNA Vaccine

mRNA vaccines carry genetic information encoding viral antigens, but they do not integrate with host cell genomes or interact with DNA, and therefore do not cause mutational risk to the host. Also, mRNA vaccines do not contain viral particles. Therefore, the mRNA vaccine does not induce the disease it prevents. In recent years, significant progress has been made in the mRNA therapy (including vaccine) technology, the modification of specific nucleosides in the mRNA sequence, and the development of various RNA packaging and introduction systems have greatly promoted the development of the mRNA vaccines^{[26]}. Much evidence shows that, compared with DNA vaccines, which are also nucleic acid vaccines, mRNA not only mediates better transfection efficiency and longer protein expression time, but also has significant advantages because it does not need to enter the nucleus to perform a function.

The mRNA vaccine can also be used as an effective means of preventing influenza virus infection. Traditional influenza vaccines generally consist of proteins found in influenza viruses, and these proteins can “train” a patient's immune system to develop mechanisms to fight influenza virus infection. However, influenza viruses mutate very quickly, often altering these proteins and rendering vaccines ineffective. That is why the influenza vaccines change every year, but do not always keep people from getting sick. The use of the mRNA vaccines to fight influenza has significant advantages over traditional vaccines. In a recent study, mRNA vaccines against H7N9 and H10N8 influenza A induced robust humoral immune responses and were well tolerated^{[27]}.

In the present invention, the anti-influenza virus siRNA molecule is combined with the influenza virus mRNA vaccine, which can effectively prevent and treat various influenza A virus infection. Since siRNA and mRNA are both RNA molecules and are only different in length and single and double strands, both can be encapsulated with the same type of nano-introduction carrier to prepare a mixed nano-drug formulation, which has broad application prospects in clinical treatment.

In one embodiment, the influenza virus mRNA vaccine designed based on the HA gene sequence was combined with a siRNA molecule for inhibiting M1 protein (sense strand is 5′-UACGCUGCAGUCCUCGCUCACUGGG-3′). The antiviral immunity of the body was activated, the antiviral immune cell viability was enhanced, and the expression of M1 gene was inhibited at the same time.

In another embodiment, the influenza virus mRNA vaccine designed based on the viral nucleoprotein NP gene sequence was combined with PB2-11 siRNA (sense strand is 5′-GAAACGAAAACGGGACUCUAGCAUA-3′) for inhibiting PB2 proteins.

Conjugation of Anti-Influenza siRNA Molecules to Small Molecule Compound

The conjugation of siRNA to nucleic acid molecules having different functions, and nucleic acid-based small molecules can increase endogenous properties and improve silencing efficiency and inhibiting rate. In one study, the siRNA of PR8-M1 was linked to a ribozyme-catalyzed degrading nucleic acid sequence to form a siRNA-ribozyme chimera, so that the ability of the siRNA to degrade nucleic acid was further enhanced, and the silencing efficiency of the optimized siRNA was improved by four times^{[30]}. Through another study, it has been found that after adding a sequence (5′-UGUGU-3′) having immunostimulatory functions to the 5′ end of NP-siRNA, the inhibiting rate for influenza viruses reached 80%, which was four times the inhibiting efficiency of siRNA alone^{[31]}. Construction of NP-1496 siRNA into a carrier containing endogenous microRNA (miRNA) (forming shRNAmir-NP) significantly enhanced its endogenous properties. After infection with a PR8 live virus strain, the NP proteins were completely inhibited, and the virus titer was reduced to about 1/100 of the control^{[32]}. The coupling between these same types of molecules can effectively provide the ability to resist influenza virus infection and replication, but is also limited by the similarity between the molecules and cannot exert the maximum synergistic effect.

The present invention comprises a novel compound molecule formed by covalently coupling the anti-influenza virus siRNA molecule and the anti-influenza A small molecule compound. The siRNA molecules include the siRNA molecules in Table 1. The anti-influenza A small molecule compound includes, but is not limited to, specific influenza virus inhibitors (M2 ion channel blockers, NA inhibitors, PA inhibitors and PB2 inhibitors) and broad-spectrum antiviral drugs. The siRNA molecule and the anti-influenza A small molecule compound can be directly linked through respective active groups, or coupled through the active groups of the linker introduced. The active groups include, but are not limited to, amino groups, carboxyl groups, hydroxyl groups, phosphate groups, epoxy groups, aldehyde groups, isocyanate groups, etc. Covalent bonds can be formed between the active groups through addition reactions, polymerization reactions, condensation reactions, etc.

In one embodiment, by condensing the phosphate groups and the hydroxyl groups into phosphate esters, Zanamivir was blocked in the siRNA molecule, or the Zanamvir was linked to the end of siRNA through the linker (such as polyethylene glycol).

In another embodiment, the amino group at one end of the linker reacted with the carboxyl group of Peramivir to form an amide bond, and the hydroxyl group at the other end of the linker reacted with the phosphate group of the siRNA to form the phosphate esters.

In another embodiment, the hydroxyl group at one end of the linker reacted with the phosphate group at the end of the siRNA, and the epoxy group at the other end of the linker was subjected to a nucleophilic reaction with the amino group of Oseltamivir.

In another embodiment, the epoxy group at one end of the linker reacted with the amino group of NA inhibitor A-192558, and the hydroxyl group at the other end of the linker reacted with the phosphate group of the siRNA. Nano-introduction (delivery) carrier and nano-drug formulation

A pharmaceutically acceptable carrier is used as an introduction (delivery) system for a siRNA drug or a composition based on the siRNA drug, and the pharmaceutically acceptable carriers generally include saline water, saccharides, polypeptides, polymers, lipids, creams, gels, micellar materials and metal nanoparticles. In one embodiment, the carrier was a histidine-lysine polymer (high-molecular polymer) described in several patents of U.S. Pat. No. 7,070,807 B2, 7,163,695 B2, and 7772201 B2, and the entire content is incorporated herein by reference. Preferably, the HKP carrier is H3K4b, H3K(+H)4b, H2K4b or H3K(+N)4b. These HKPs have a lysine backbone, and four branches of the lysine backbone contain multiple repeated histidine, lysine or asparagine.

In one embodiment, HKP was H3K4b with the following structure:
(R)K(R)-K(R)-(R)K(X), where R=KHHHKHHHKHHHKHHHK or R=KHHHKHHHNHHHNHHHN, X=C(0)NH2, K=lysine, H=histidine, and N=asparagine.

In another embodiment, HKP was H3K(+H)4b with the following structure:
(R)K(R)-K(R)-(R)K(X), where R=KHHHKHHHKHHHHKHHHK, X=C(0)NH2, K=lysine, and H=histidine.

In another embodiment, HKP was H2K4b with the following structure:
(R)K(R)-K(R)-(R)K(X), where R=KHKHHKHHKHHKHHKHHKHK, X=C(0)NH2, K=lysine, and H=histidine.

In another embodiment, HKP was H3K(+N)4b with the following structure:
(R)K(R)-K(R)-(R)K(X), where R=KHHHHKHHHKHHHNHHHN, X=C(0)NH2, K=lysine, H=histidine, and N=asparagine.

Provided is the nano-drug formulation prepared from HKP or a siRNA drug or the composition based on the siRNA drug, where the HKP carries a positive charge, while siRNA, a composition of siRNA and siRNA, a composition of siRNA and mRNA vaccines, etc. carry a negative charge. When an HKP aqueous solution is mixed with the siRNA or the composition based on the siRNA drug at a specific mass ratio (such as 4:1), the nanoparticles will be formed through self-assembling. The average diameter of the nanoparticles is in the range of 30-400 nm, and further preferably, the size of the nanoparticles is 50-150 nm.

### Aerosol Inhalation Administration

The present invention also comprises a method for preventing or treating influenza A virus infection using anti-influenza virus siRNA molecules and a pharmaceutical composition based on these siRAN molecules. As stated herein, “treating” or “treatment” refers to reducing the severity of influenza A virus infection diseases or curing the influenza A virus infection diseases. A therapeutically effective dose of the composition of the present invention is administered to mammals. In one embodiment, the mammals were humans, rodents (such as rats, mice, or guinea pigs), ferrets, or non-human primates (such as monkeys). In one aspect of the embodiment, the mammals were experimental animals, such as rodents. In another aspect of the embodiment, the mammals were non-human primates, such as monkeys. In another aspect of the embodiment, the mammals were humans. As stated herein, a “therapeutically effective dose” is a dose that prevent or reduce the severity of influenza A virus infection, or cure the influenza A virus infection.

In one embodiment, a therapeutically effective dose of the pharmaceutical composition administered to humans included the siRNA molecules ranging from about 0.1 mg per kilogram of human body weight to about 10 mg per kilogram of human body weight.

In another aspect of the embodiment, a therapeutically effective dose of the pharmaceutical composition administered to humans included the siRNA molecular composition ranging from about 0.1 mg per kilogram of human body weight to about 100 mg per kilogram of human body weight.

In view of the administration included herein, the route of administration can be determined by those skilled in the technology. These routes include intranasal administration, airway instillation, and inhalation administration, for example, by use of an atomizing spray device. In some embodiments, routes of administration also included injection instillation and intraperitoneal, intravenous, intradermal, intravaginal, and subcutaneous administration. Preferably, the nano-drug formulation is delivered to the virus-infected lower respiratory tract or the lung by inhalation administration or intravenous injection. Further preferably, the drug formulation is introduced into the virus-infected lower respiratory tract or the lung by aerosol inhalation administration.

### REFERENCES

1. Bouvier N M, Palese P. The biology of influenza viruses. Vaccination with the vaccine. 2008; 26 (Suppl. 4):D49-53.
2. Jagger B W, Wise H M, Kash J C, Walters K A, Wills N M, Xiao Y L, et al. An overlapping protein-coding region in influenza A virus segment 3 modulates the host response. Science. 2012; 337 (6091):199-204.
3. Tong S, Zhu X, L1 Yong, Shi M, Zhang Jie, Bourgeois M, et al. New world bats harbor diverse influenza A viruses. PLoS Pathog. 2013; 9(10):e1003657.
4. Emily Rumschlag-Booms, Rong Linum. Influenza A virus invasion: implications in virulence and future therapeutics. Adv Virol. 2013; 2013:121924.
5. Takashita E, Fujisaki S, Yokoyama M, Shirakura M, Morita H, Nakamura K, et al. In vitro characterization of multidrug-resistant influenza A (H1N1) pdm09 viruses carrying a dual neuraminidase mutation isolated from immunocompromised patients. Pathogens. 2020; 9(9):725.
6. Bright R A, Medina M J, Xu X, et al. Incidence of adamantane resistance among influenza A (H3N2) viruses isolated worldwide from 1994 to 2005: a cause for concern. Lancet. 2005; 366:1175-1181.
7. Goldhill D H, Te Velthuis A J W, Fletcher R A, Langat P, Zambon M, Lackenby A, Barclay W S. The mechanism of resistance to Favipiravir in influenza virus. Proc Natl Acad Sci U.S.A. 2018; 115 (45):11613-11618.
8. Jefferson T, Jones M, Doshi P, Del Mar C, Dooley L, Foxlee R. Neuraminidase inhibitors for preventing and treating influenza in healthy adults. Cochrane Database Syst Rev. 2012; (2):CD001265.
9. Chan-Tack K M, Murray J S, Birnkrant D B. Use of Ribavirin to treat influenza. N Engl J Med. 2009; 361:1713-1714.
10. Beigel J H, Bao Y, Beeler J, et al. Oseltamivir, Amantadine, and Ribavirin combination antiviral therapy versus Oseltamivir monotherapy for the treatment of influenza: a multicentre, double-blind, randomised phase 2 trial. Lancet Infect Dis. 2017; 17:1255-1265.
11. Furuta Y, Komeno T, Nakamura T. Favipiravir (T-705), a broad spectrum inhibitor of viral RNA polymerase. Proc Natl Acad Sci U.S.A. 2017; 93:449-463.
12. Sangawa H, Komeno T, Nishikawa H, Yoshida A, Takahashi K, Nomura N, and Furuta Y. Mechanism of action of T-705 ribosyl triphosphate against influenza virus RNA polymerase. Antimicrob Agents Chemother. 2013; 57:5202-5208.
13. Mollaie H R, Monavari S H, Arabzadeh S A, Shamsi-Shahrabadi M, Fazlalipour M, et al. RNAi and miRNA in viral infections and cancers. Asian Pac J Cancer Prev. 2013; 14 (12):7045-7056.
14. Joo J Y, Yhee J Y, Kim S H, Kim K. The potential and advances in RNAi therapy: chemical and structural modifications of siRNA molecules and use of biocompatible nanocarriers. J Control Release. 2014; 193:113-121.
15. Barik S. siRNA for influenza therapy. Viruses. 2010; 2:1448-1457.
16. Qing Ge, Michael D M, Tam N, Shen Q H, Phillip A S, Herman N E, Chen J Z. RNA interference of influenza virus production by directly targeting mRNA for degradation and indirectly inhibiting all viral RNA transcription. Proc Natl Acad Sci U.S.A. 2003; 100 (5):2718-23.
17. Bhawana J, Amita J, Om P, Ajay K S, Tanushree D, Mastan S, Singh L P. In vitro validation of self designed “universal human Influenza A siRNA”. Indian J Exp Biol. 2015; 53 (8):514-521.
18. Stephen M K, Chia Y L, Terrence M T, Suzanne L E. Protection against lethal influenza virus challenge by RNA interference in vivo. Proc Natl Acad Sci U.S.A. 2004; 101 (23):8682-8686.
19. Roopali R, Madhu K, Prashant K, Binod K, Sonal S, Neha G, Latika S. Small interfering RNA targeting the nonstructural gene 1 transcript inhibits influenza A virus replication in experimental mice. 2012; 22 (6):414-422.
20. Neumann G, Noda T, Kawaoka Y. Emergence and pandemic potential of swine-origin HiN1 influenza virus. Nature. 2009; 459:931-939.
21. Tarbet E B, Vollmer A H, Hurst B L, Barnard D L, Furuta Y, Smee D F. In vitro activity of Favipiravir and neuraminidase inhibitor combinations against oseltamivir-sensitive and oseltamivir-resistant pandemic influenza A (H1N1) virus. Arch Virol. 2014; 159:1279-1291.
22. Marathe B M, Wong S S, Vogel P, Garcia-Alcalde F, Webster R G, Webby R J, Najera I, Govorkova W A. Combinations of Oseltamivir and T-705 extend the treatment window for highly pathogenic influenza A(H5N1) virus infection in mice. Sci Rep. 2016; 626742.
23. Kiso M, Lopes TJS, Yamayoshi S, Nakajima N, Hasegawa H, Neumann G, Kawaoka Y. Combination therapy with neuraminidase and polymerase inhibitors in nude mice infected with influenza virus. J Infect Dis. 2018; 217:887-896.
24. Wang Y, Fan G, Salam A, Horby P, Hayden F G, Chen C, Pan Jie, Zheng Jie, Lu B, Guo L, et al. Comparative effectiveness of combined Favipiravir and Oseltamivir therapy versus Oseltamivir monotherapy in critically ill patients with influenza virus infection. J Infect Dis. 2020; 221 (10):1688-1698.
25. Aurica G T, Vasile Laza, Michael R E, James A H, Hongwei W, Nathan W B, Patrick M, Mihnea T Z, Tatiana K, Anthony J C, et al. RSV-induced bronchial epithelial cell PD-L1 expression inhibits CD81T cell nonspecific antiviral activity. J Infect Dis. 2011; 203:85-94.
26. Norbert P, Michael J H, Frederick W P, Drew W. RNA vaccines—a new era in vaccinology. Nat Rev Drug Discov. 2018; 17 (4):261-279.
27. Robert A F, Rainard F, Igor S, Amilcar M R, Lori P, Mike W, Joseph J S, et al. mRNA vaccines against H10N8 and H7N9 influenza viruses of pandemic potential are immunogenic and well tolerated in healthy adults in phase 1 randomized clinical trials. Vaccine. 2019; 37 (25):3326-3334.
28. Trehanpati N, Vyas A K. Immune regulation by T regulatory cells in hepatitis B virus-related inflammation and cancer. Scand J Immunol. 2017; 85:175-181.
29. Qian Zhang, Mu X, Dong H, Hu G, Zhang T, He C, Naila S. Pulmonary endothelium-derived PD-L1 induced by the H9N2 avian influenza virus inhibits the immune response of T cells. Virol J. 2020; 17(1): 92.
30. Prashant K, Vikas S, Rajesh V, Nidhi G, Akhil C B, Madhu K. Potent inhibition of influenza virus replication with novel siRNA-chimeric-ribozyme constructs. Antiviral Res. 2010; 87 (2):204-212.
31. Gaurav J, Paban K D, Ankita A, Shashi S, Manmohan P. Bifunctional siRNA containing immunostimulatory motif enhances protection. Curr Gene Ther. 2015; 15 (5):492-502.
32. Xu F, Liu G Q, Liu Q, Zhou Y. RNA interference of influenza A virus replication by microRNA-adapted lentiviral loop short hairpin RNA. J Gen Virol. 2015; 96 (10):2971-2981.

1. A siRNA molecule for inhibiting replication of the influenza virus, wherein the siRNA molecule includes a sense strand and an antisense strand, the sequence of the sense strand is selected from any one of SEQ ID Nos. 1-16, SEQ ID No. 20-54, SEQ ID No. 56-69, SEQ ID No. 71-91, SEQ ID No. 93, and SEQ ID No. 94, and the antisense strand is selected from any one of SEQ ID No. 98-113, SEQ ID No. 117-151, SEQ ID No. 153-166, SEQ ID No. 168-188, SEQ ID No. 190, and SEQ ID No. 191, which is complementary to the sense strand.

2. A siRNA drug for preventing or treating influenza virus infection, wherein the siRNA drug includes active ingredient(s), and the active ingredient(s) include one or more of the siRNA molecules of claim 1.

3. The siRNA drug for preventing or treating influenza virus infection of claim 2, wherein the active ingredient(s) further include one or more other siRNA molecule(s) for inhibiting replication of the influenza virus; preferably, the sequence of the sense strand of the other siRNA molecules for inhibiting replication of the influenza virus is selected from any one of SEQ ID No. 17-19, SEQ ID No. 55, SEQ ID No. 70, SEQ ID No. 92, SEQ ID No. 95-97, and the antisense strand of other siRNA molecules for inhibiting replication of the influenza virus is selected from any one of SEQ ID No. 114-116, SEQ ID No. 152, SEQ ID No. 167, SEQ ID No. 189, and SEQ ID No. 192-194, which is complementary to the sense strands of the other siRNA molecules for inhibiting replication of the influenza virus.

4. A pharmaceutical composition for preventing or treating influenza virus infection, wherein the active ingredients of the pharmaceutical composition include a siRNA molecule for inhibiting replication of the influenza virus and the other molecule(s), and the other molecule(s) include one or more of a siRNA molecule for inhibiting PD-1 expression, a siRNA molecule for inhibiting the expression of PD-L1, anti-influenza virus small molecule compound, an influenza mRNA vaccine, or a monoclonal antibody for resisting the influenza viruses.

5. The pharmaceutical composition for preventing or treating influenza virus infection of claim 4, wherein the siRNA molecules for inhibiting replication of the influenza virus are designed for conserved gene sequences for different strains of influenza A virus, including one or more of H1N1, H5N1, H7N9, or H3N2 subtypes; the siRNA molecules for inhibiting replication of the influenza virus block the life cycle of virus replication by targeting and inhibiting the expression of key genes related to invasion, replication, assembly or release of the influenza virus, reduce viral titers, and inhibit infection until complete virus clearance.

6. The pharmaceutical composition for preventing or treating influenza virus infection of claim 5, wherein the siRNA molecule(s) for inhibiting replication of the influenza virus are selected from one or more of the following siRNA molecules: the sense strand is selected from any one of SEQ ID Nos. 1-97, and the antisense strand is selected from any one of SEQ ID Nos. 98-194, which is complementary to the sense strand.

7. The pharmaceutical composition for preventing or treating influenza virus infection of claim 4, wherein the anti-influenza virus small molecule compounds are specific influenza virus inhibitors and/or broad-spectrum antiviral small molecule compounds, preferably, the specific influenza virus inhibitor(s) are selected from one or more of M2 ion channel blockers, NA inhibitors, PA inhibitors, and PB2 inhibitors, and the broad-spectrum antiviral small molecule compound(s) are selected from one or more of Ribavirin, Nitazoxanide, Arbidol hydrochloride, Favipiravir, Zanamivir, and Peramivir.

8. The pharmaceutical composition for preventing or treating influenza virus infection of claim 4, wherein the siRNA molecule for inhibiting replication of the influenza virus is linked to the anti-influenza virus small molecule compound through respective active groups, or is coupled with the anti-influenza virus small molecule compound through the active groups of the linker introduced into the siRNA molecule for inhibiting replication of the influenza virus; and the reactive groups comprise one or more of amino groups, carboxyl groups, hydroxyl groups, phosphate groups, epoxy groups, aldehyde groups and isocyanate groups.

9. The pharmaceutical composition for preventing or treating influenza virus infection of claim 4, wherein the siRNA molecule for inhibiting PD-1 expression is designed based on the homologous sequences between a human PD-1 gene and a mouse PD-1 gene, and the siRNA molecule for inhibiting the PD-L1 expression is designed based on the homologous sequences between a human PD-L1 gene and a mouse PD-L1 gene, and preferably, the homologous sequences refer to the DNA sequences that are confirmed to be 100% identical after the two genes of humans and mice are aligned;
the influenza mRNA vaccine is a messenger ribonucleic acid vaccine designed based on the influenza virus gene sequences, preferably, the influenza virus gene is a gene encoding a viral structural protein and/or a non-structural protein. Further preferably, the gene encoding the viral structural protein(s) are selected from one or more of PB2, PB1, PA, HA, NP, NA, M1, or M2, and the genes encoding the non-structural protein are NS1 and/or NS2; and
the anti-influenza virus small molecule compounds are specific influenza virus inhibitors and/or broad-spectrum antiviral small molecule compounds, preferably, the specific influenza virus inhibitor(s) are selected from one or more of M2 ion channel blockers, NA inhibitors, PA inhibitors, and PB2 inhibitors, and the broad-spectrum antiviral small molecule compound(s) are selected from one or more of Ribavirin, Nitazoxanide, Arbidol hydrochloride, and Favipiravir.

10. The pharmaceutical composition for preventing or treating influenza virus infection of claim 4, wherein the siRNA molecule(s) for inhibiting the PD-1 expression are selected from one or more of the following siRNA molecules: the sequence of the sense strand is selected from any one of SEQ ID Nos. 195-206, and the antisense strand is selected from any one of SEQ ID Nos. 207-218, which is complementary to the sense strand; and
the siRNA(s) for inhibiting PD-L1 expression are selected from one or more of the following siRNA molecules: the sequence of the sense strand is selected from any one of SEQ ID Nos. 219-230, and the antisense strand is selected from any one of SEQ ID Nos. 231-242, which is complementary to the sense strand.

11. A siRNA-small molecule drug conjugate, wherein the siRNA-small molecule drug conjugate is formed by covalent bond coupling of the siRNA molecule for inhibiting replication of the influenza virus and the anti-influenza small molecule drug.

12. An application of the siRNA-small molecule drug conjugate of claim 11 to the preparation of a drug for preventing or treating influenza virus infection.

13. The siRNA drug for preventing or treating influenza virus infection of claim 2, wherein a formulation is prepared from the siRNA drug for preventing or treating influenza virus infection, the pharmaceutical composition for preventing or treating influenza virus infection, or the siRNA-small molecule drug conjugate and the pharmaceutically acceptable carrier(s), the pharmaceutically acceptable carrier(s) are selected from one or more of saline water, saccharides, polypeptides, high molecular polymers, lipids, creams, gels, micellar materials, or metal nanoparticles, and preferably, the high-molecular polymers are the polypeptide high-molecular polymers.

14. The siRNA drug of claim 13, wherein the formulation is a nano-drug formulation; and the dosage form of the nano-drug formulation is an oral formulation, injection, or aerosol inhalation formulation.

15. The siRNA drug for preventing or treating influenza virus infection of claim 2, wherein the influenza virus(es) are one or more of the G4 EA H1N1 virus strain, the H1N1 virus strain, the H5N1 virus strain, the H7N9 virus strain, or the H3N2 virus strain.

16. The pharmaceutical composition for preventing or treating influenza virus infection of claim 4, wherein a formulation is prepared from the siRNA drug for preventing or treating influenza virus infection, the pharmaceutical composition for preventing or treating influenza virus infection, or the siRNA-small molecule drug conjugate and the pharmaceutically acceptable carrier(s), the pharmaceutically acceptable carrier(s) are selected from one or more of saline water, saccharides, polypeptides, high molecular polymers, lipids, creams, gels, micellar materials, or metal nanoparticles, and preferably, the high-molecular polymers are the polypeptide high-molecular polymers.

17. The pharmaceutical composition of claim 16, wherein the formulation is a nano-drug formulation; and the dosage form of the nano-drug formulation is an oral formulation, injection, or aerosol inhalation formulation.

18. The pharmaceutical composition for preventing or treating influenza virus infection of claim 4, wherein the influenza virus(es) are one or more of the G4 EA H1N1 virus strain, the H1N1 virus strain, the H5N1 virus strain, the H7N9 virus strain, or the H3N2 virus strain.

19. The application of claim 12, wherein the influenza virus(es) are one or more of the G4 EA H1N1 virus strain, the H1N1 virus strain, the H5N1 virus strain, the H7N9 virus strain, or the H3N2 virus strain.


SEQUENCE LISTING (242 sequences)

SEQ ID NO: 1 - RNA, 19 nt, Artificial Sequence
  the sequence is synthesized
guggaccauauggccauaa

SEQ ID NO: 2 - RNA, 19 nt, Artificial Sequence
  the sequence is synthesized
cgggacucuagcauacuua

SEQ ID NO: 3 - RNA, 19 nt, Artificial Sequence
  the sequence is synthesized
gacucuagcauacuuacug

SEQ ID NO: 4 - RNA, 19 nt, Artificial Sequence
  the sequence is synthesized
cucuagcauacuuacugac

SEQ ID NO: 5 - RNA, 19 nt, Artificial Sequence
  the sequence is synthesized
cuagcauacuuacugacag

SEQ ID NO: 6 - RNA, 19 nt, Artificial Sequence
  the sequence is synthesized
gcauacuuacugacagcca

SEQ ID NO: 7 - RNA, 19 nt, Artificial Sequence
  the sequence is synthesized
cuuacugacagccagacag

SEQ ID NO: 8 - RNA, 19 nt, Artificial Sequence
  the sequence is synthesized
gccagacagcgaccaaaag

SEQ ID NO: 9 - RNA, 19 nt, Artificial Sequence
  the sequence is synthesized
gcgaccaaaagaauucgga

SEQ ID NO: 10 - RNA, 19 nt, Artificial Sequence
  the sequence is synthesized
gaauucggauggccaucaa

SEQ ID NO: 11 - RNA, 25 nt, Artificial Sequence
  the sequence is synthesized
gaaacgaaaacgggacucuagcaua

SEQ ID NO: 12 - RNA, 25 nt, Artificial Sequence
  the sequence is synthesized
cgaaaacgggacucuagcauacuua

SEQ ID NO: 13 - RNA, 25 nt, Artificial Sequence
  the sequence is synthesized
gcauacuuacugacagccagacagc

SEQ ID NO: 14 - RNA, 25 nt, Artificial Sequence
  the sequence is synthesized
cuuacugacagccagacagcgacca

SEQ ID NO: 15 - RNA, 25 nt, Artificial Sequence
  the sequence is synthesized
gacagccagacagcgaccaaaagaa

SEQ ID NO: 16 - RNA, 25 nt, Artificial Sequence
  the sequence is synthesized
ccaaaagaauucggauggccaucaa

SEQ ID NO: 17 - RNA, 25 nt, Artificial Sequence
  the sequence is synthesized
auggccauccgaauucuuuuggucg

SEQ ID NO: 18 - RNA, 25 nt, Artificial Sequence
  the sequence is synthesized
uugauggccauccgaauucuuuugg

SEQ ID NO: 19 - RNA, 19 nt, Artificial Sequence
  the sequence is synthesized
gggaacagauguacacucc

SEQ ID NO: 20 - RNA, 19 nt, Artificial Sequence
  the sequence is synthesized
cacauucccuuauacugga

SEQ ID NO: 21 - RNA, 19 nt, Artificial Sequence
  the sequence is synthesized
ccuccauacagccauggaa

SEQ ID NO: 22 - RNA, 19 nt, Artificial Sequence
  the sequence is synthesized
cagccauggaacaggaaca

SEQ ID NO: 23 - RNA, 19 nt, Artificial Sequence
  the sequence is synthesized
ccauggaacaggaacagga

SEQ ID NO: 24 - RNA, 19 nt, Artificial Sequence
  the sequence is synthesized
gaacaggaacaggauacac

SEQ ID NO: 25 - RNA, 19 nt, Artificial Sequence
  the sequence is synthesized
gaugaugggcauguucaac

SEQ ID NO: 26 - RNA, 19 nt, Artificial Sequence
  the sequence is synthesized
guggaggccauggugucua

SEQ ID NO: 27 - RNA, 19 nt, Artificial Sequence
  the sequence is synthesized
gagaucaugaagaucuguu

SEQ ID NO: 28 - RNA, 19 nt, Artificial Sequence
  the sequence is synthesized
gaucaugaagaucuguucc

SEQ ID NO: 29 - RNA, 19 nt, Artificial Sequence
  the sequence is synthesized
caugaagaucuguuccacc

SEQ ID NO: 30 - RNA, 19 nt, Artificial Sequence
  the sequence is synthesized
gaagaucuguuccaccauu

SEQ ID NO: 31 - RNA, 25 nt, Artificial Sequence
  the sequence is synthesized
cuuauacuggagauccuccauacag

SEQ ID NO: 32 - RNA, 25 nt, Artificial Sequence
  the sequence is synthesized
cuggagauccuccauacagccaugg

SEQ ID NO: 33 - RNA, 25 nt, Artificial Sequence
  the sequence is synthesized
gccauggaacaggaacaggauacac

SEQ ID NO: 34 - RNA, 25 nt, Artificial Sequence
  the sequence is synthesized
caugguggaggccauggugucuagg

SEQ ID NO: 35 - RNA, 25 nt, Artificial Sequence
  the sequence is synthesized
gaucaugaagaucuguuccaccauu

SEQ ID NO: 36 - RNA, 25 nt, Artificial Sequence
  the sequence is synthesized
caugaagaucuguuccaccauugaa

SEQ ID NO: 37 - RNA, 19 nt, Artificial Sequence
  the sequence is synthesized
gggauuccuuucgucaguc

SEQ ID NO: 38 - RNA, 19 nt, Artificial Sequence
  the sequence is synthesized
gauuccuuucgucaguccg

SEQ ID NO: 39 - RNA, 19 nt, Artificial Sequence
  the sequence is synthesized
gccugauuaaugaucccug

SEQ ID NO: 40 - RNA, 19 nt, Artificial Sequence
  the sequence is synthesized
cugauuaaugaucccuggg

SEQ ID NO: 41 - RNA, 19 nt, Artificial Sequence
  the sequence is synthesized
gauuaaugaucccuggguu

SEQ ID NO: 42 - RNA, 19 nt, Artificial Sequence
  the sequence is synthesized
gaucccuggguuuugcuua

SEQ ID NO: 43 - RNA, 19 nt, Artificial Sequence
  the sequence is synthesized
cuggguuuugcuuaaugca

SEQ ID NO: 44 - RNA, 19 nt, Artificial Sequence
  the sequence is synthesized
cuuaaugcaucuugguuca

SEQ ID NO: 45 - RNA, 19 nt, artificial sequence
  the sequence is synthesized
gcaucuugguucaacuccu

SEQ ID NO: 46 - RNA, 19 nt, Artificial Sequence
  the sequence is synthesized
cuugguucaacuccuuccu

SEQ ID NO: 47 - RNA, 19 nt, Artificial Sequence
  the sequence is synthesized
guucaacuccuuccucaca

SEQ ID NO: 48 - RNA, 19 nt, Artificial Sequence
  the sequence is synthesized
cuccuuccucacacaugca

SEQ ID NO: 49 - RNA, 25 nt, Artificial Sequence
  the sequence is synthesized
gaaagaauauggggaagauccgaaa

SEQ ID NO: 50 - RNA, 25 nt, Artificial Sequence
  the sequence is synthesized
gccugauuaaugaucccuggguuuu

SEQ ID NO: 51 - RNA, 25 nt, Artificial Sequence
  the sequence is synthesized
cuuaaugcaucuugguucaacuccu

SEQ ID NO: 52 - RNA, 25 nt, Artificial Sequence
  the sequence is synthesized
gcaucuugguucaacuccuuccuca

SEQ ID NO: 53 - RNA, 25 nt, Artificial Sequence
  the sequence is synthesized
cuugguucaacuccuuccucacaca

SEQ ID NO: 54 - RNA, 25 nt, Artificial Sequence
  the sequence is synthesized
guucaacuccuuccucacacaugca

SEQ ID NO: 55 - RNA, 19 nt, Artificial Sequence
  the sequence is synthesized
gcaauugaggagugccuga

SEQ ID NO: 56 - RNA, 19 nt, Artificial Sequence
  the sequence is synthesized
gagucuucgagcucucgga

SEQ ID NO: 57 - RNA, 19 nt, Artificial Sequence
  the sequence is synthesized
cuucgagcucucggacgaa

SEQ ID NO: 58 - RNA, 19 nt, Artificial Sequence
  the sequence is synthesized
cgagcucucggacgaaaag

SEQ ID NO: 59 - RNA, 19 nt, Artificial Sequence
  the sequence is synthesized
gcucucggacgaaaaggca

SEQ ID NO: 60 - RNA, 19 nt, Artificial Sequence
  the sequence is synthesized
cucggacgaaaaggcaacg

SEQ ID NO: 61 - RNA, 19 nt, Artificial Sequence
  the sequence is synthesized
cggacgaaaaggcaacgaa

SEQ ID NO: 62 - RNA, 19 nt, Artificial Sequence
  the sequence is synthesized
cgaaaaggcaacgaacccg

SEQ ID NO: 63 - RNA, 19 nt, Artificial Sequence
  the sequence is synthesized
gaaaaggcaacgaacccga

SEQ ID NO: 64 - RNA, 19 nt, Artificial Sequence
  the sequence is synthesized
ccuuugacaugaguaauga

SEQ ID NO: 65 - RNA, 19 nt, Artificial Sequence
  the sequence is synthesized
cuuauuucuucggagacaa

SEQ ID NO: 66 - RNA, 25 nt, Artificial Sequence
  the sequence is synthesized
gagucuucgagcucucggacgaaaa

SEQ ID NO: 67 - RNA, 25 nt, Artificial Sequence
  the sequence is synthesized
cgaaaaggcaacgaacccgaucgug

SEQ ID NO: 68 - RNA, 25 nt, Artificial Sequence
  the sequence is synthesized
gaaaaggcaacgaacccgaucgugc

SEQ ID NO: 69 - RNA, 25 nt, Artificial Sequence
  the sequence is synthesized
cuuauuucuucggagacaaugcaga

SEQ ID NO: 70 - RNA, 19 nt, Artificial Sequence
  the sequence is synthesized
cuccgaagaaauaagaucc

SEQ ID NO: 71 - RNA, 19 nt, Artificial Sequence
  the sequence is synthesized
gucuuggccagacggugcu

SEQ ID NO: 72 - RNA, 19 nt, Artificial Sequence
  the sequence is synthesized
ggccagacggugcugaguu

SEQ ID NO: 73 - RNA, 19 nt, Artificial Sequence
  the sequence is synthesized
gacggugcugaguugccau

SEQ ID NO: 74 - RNA, 25 nt, Artificial Sequence
  the sequence is synthesized
ggccagacggugcugaguugccauu

SEQ ID NO: 75 - RNA, 19 nt, Artificial Sequence
  the sequence is synthesized
gucuucuaaccgaggucga

SEQ ID NO: 76 - RNA, 19 nt, Artificial Sequence
  the sequence is synthesized
cuucuaaccgaggucgaaa

SEQ ID NO: 77 - RNA, 19 nt, Artificial Sequence
  the sequence is synthesized
cuaaccgaggucgaaacgu

SEQ ID NO: 78 - RNA, 19 nt, Artificial Sequence
  the sequence is synthesized
ccgaggucgaaacguacgu

SEQ ID NO: 79 - RNA, 19 nt, Artificial Sequence
  the sequence is synthesized
cgaggucgaaacguacguu

SEQ ID NO: 80 - RNA, 19 nt, Artificial Sequence
  the sequence is synthesized
cccucaaagccgagaucgc

SEQ ID NO: 81 - RNA, 19 nt, Artificial Sequence
  the sequence is synthesized
ggcuaaagacaagaccaau

SEQ ID NO: 82 - RNA, 19 nt, Artificial Sequence
  the sequence is synthesized
cacgcucaccgugcccagu

SEQ ID NO: 83 - RNA, 19 nt, Artificial Sequence
  the sequence is synthesized
cgcucaccgugcccaguga

SEQ ID NO: 84 - RNA, 19 nt, Artificial Sequence
  the sequence is synthesized
cgugcccagugagcgagga

SEQ ID NO: 85 - RNA, 19 nt, Artificial Sequence
  the sequence is synthesized
ccagugagcgaggacugca

SEQ ID NO: 86 - RNA, 19 nt, Artificial Sequence
  the sequence is synthesized
gagcgaggacugcagcgua

SEQ ID NO: 87 - RNA, 19 nt, Artificial Sequence
  the sequence is synthesized
ggacugcagcguagacgcu

SEQ ID NO: 88 - RNA, 25 nt, Artificial Sequence
  the sequence is synthesized
gucuucuaaccgaggucgaaacgua

SEQ ID NO: 89 - RNA, 25 nt, Artificial Sequence
  the sequence is synthesized
cacgcucaccgugcccagugagcga

SEQ ID NO: 90 - RNA, 25 nt, Artificial Sequence
  the sequence is synthesized
gcucaccgugcccagugagcgagga

SEQ ID NO: 91 - RNA, 25 nt, Artificial Sequence
  the sequence is synthesized
ccgugcccagugagcgaggacugca

SEQ ID NO: 92 - RNA, 25 nt, Artificial Sequence
  the sequence is synthesized
cccagugagcgaggacugcagcgua

SEQ ID NO: 93 - RNA, 25 nt, Artificial Sequence
  the sequence is synthesized
cagugagcgaggacugcagcguaga

SEQ ID NO: 94 - RNA, 25 nt, Artificial Sequence
  the sequence is synthesized
gacugcagcguagacgcuuugucca

SEQ ID NO: 95 - RNA, 19 nt, Artificial Sequence
  the sequence is synthesized
uucgaccucgguuagaaga

SEQ ID NO: 96 - RNA, 25 nt, Artificial Sequence
  the sequence is synthesized
uacgcugcaguccucgcucacuggg

SEQ ID NO: 97 - RNA, 19 nt, Artificial Sequence
  the sequence is synthesized
auccacagcauucugcugu

SEQ ID NO: 98 - RNA, 19 nt, Artificial Sequence
  the sequence is synthesized
uuauggccauaugguccac

SEQ ID NO: 99 - RNA, 19 nt, Artificial Sequence
  the sequence is synthesized
uaaguaugcuagagucccg

SEQ ID NO: 100 - RNA, 19 nt, Artificial Sequence
  the sequence is synthesized
caguaaguaugcuagaguc

SEQ ID NO: 101 - RNA, 19 nt, Artificial Sequence
  the sequence is synthesized
gucaguaaguaugcuagag

SEQ ID NO: 102 - RNA, 19 nt, Artificial Sequence
  the sequence is synthesized
cugucaguaaguaugcuag

SEQ ID NO: 103 - RNA, 19 nt, Artificial Sequence
  the sequence is synthesized
uggcugucaguaaguaugc

SEQ ID NO: 104 - RNA, 19 nt, Artificial Sequence
  the sequence is synthesized
cugucuggcugucaguaag

SEQ ID NO: 105 - RNA, 19 nt, Artificial Sequence
  the sequence is synthesized
cuuuuggucgcugucuggc

SEQ ID NO: 106 - RNA, 19 nt, Artificial Sequence
  the sequence is synthesized
uccgaauucuuuuggucgc

SEQ ID NO: 107 - RNA, 19 nt, Artificial Sequence
  the sequence is synthesized
uugauggccauccgaauuc

SEQ ID NO: 108 - RNA, 25 nt, artificial sequence
  the sequence is synthesized
uaugcuagagucccguuuucguuuc

SEQ ID NO: 109 - RNA, 25 nt, Artificial Sequence
  the sequence is synthesized
uaaguaugcuagagucccguuuucg

SEQ ID NO: 110 - RNA, 25 nt, Artificial Sequence
  the sequence is synthesized
gcugucuggcugucaguaaguaugc

SEQ ID NO: 111 - RNA, 25 nt, Artificial Sequence
  the sequence is synthesized
uggucgcugucuggcugucaguaag

SEQ ID NO: 112 - RNA, 25 nt, Artificial Sequence
  the sequence is synthesized
uucuuuuggucgcugucuggcuguc

SEQ ID NO: 113 - RNA, 25 nt, Artificial Sequence
  the sequence is synthesized
uugauggccauccgaauucuuuugg

SEQ ID NO: 114 - RNA, 25 nt, Artificial Sequence
  the sequence is synthesized
cgaccaaaagaauucggauggccau

SEQ ID NO: 115 - RNA, 25 nt, artificial sequence
  the sequence is synthesized
ccaaaagaauucggauggccaucaa

SEQ ID NO: 116 - RNA, 19 nt, Artificial Sequence
  the sequence is synthesized
ggaguguacaucuguuccc

SEQ ID NO: 117 - RNA, 19 nt, Artificial Sequence
  the sequence is synthesized
uccaguauaagggaaugug

SEQ ID NO: 118 - RNA, 19 nt, Artificial Sequence
  the sequence is synthesized
uuccauggcuguauggagg

SEQ ID NO: 119 - RNA, 19 nt, Artificial Sequence
  the sequence is synthesized
uguuccuguuccauggcug

SEQ ID NO: 120 - RNA, 19 nt, Artificial Sequence
  the sequence is synthesized
uccuguuccuguuccaugg

SEQ ID NO: 121 - RNA, 19 nt, Artificial Sequence
  the sequence is synthesized
guguauccuguuccuguuc

SEQ ID NO: 122 - RNA, 19 nt, Artificial Sequence
  the sequence is synthesized
guugaacaugcccaucauc

SEQ ID NO: 123 - RNA, 19 nt, Artificial Sequence
  the sequence is synthesized
uagacaccauggccuccac

SEQ ID NO: 124 - RNA, 19 nt, Artificial Sequence
  the sequence is synthesized
aacagaucuucaugaucuc

SEQ ID NO: 125 - RNA, 19 nt, Artificial Sequence
  the sequence is synthesized
ggaacagaucuucaugauc

SEQ ID NO: 126 - RNA, 19 nt, Artificial Sequence
  the sequence is synthesized
gguggaacagaucuucaug

SEQ ID NO: 127 - RNA, 19 nt, Artificial Sequence
  the sequence is synthesized
aaugguggaacagaucuuc

SEQ ID NO: 128 - RNA, 25 nt, Artificial Sequence
  the sequence is synthesized
cuguauggaggaucuccaguauaag

SEQ ID NO: 129 - RNA, 25 nt, Artificial Sequence
  the sequence is synthesized
ccauggcuguauggaggaucuccag

SEQ ID NO: 130 - RNA, 25 nt, Artificial Sequence
  the sequence is synthesized
guguauccuguuccuguuccauggc

SEQ ID NO: 131 - RNA, 25 nt, Artificial Sequence
  the sequence is synthesized
ccuagacaccauggccuccaccaug

SEQ ID NO: 132 - RNA, 25 nt, Artificial Sequence
  the sequence is synthesized
aaugguggaacagaucuucaugauc

SEQ ID NO: 133 - RNA, 25 nt, Artificial Sequence
  the sequence is synthesized
uucaaugguggaacagaucuucaug

SEQ ID NO: 134 - RNA, 19 nt, Artificial Sequence
  the sequence is synthesized
gacugacgaaaggaauccc

SEQ ID NO: 135 - RNA, 19 nt, Artificial Sequence
  the sequence is synthesized
cggacugacgaaaggaauc

SEQ ID NO: 136 - RNA, 19 nt, Artificial Sequence
  the sequence is synthesized
cagggaucauuaaucaggc

SEQ ID NO: 137 - RNA, 19 nt, Artificial Sequence
  the sequence is synthesized
cccagggaucauuaaucag

SEQ ID NO: 138 - RNA, 19 nt, Artificial Sequence
  the sequence is synthesized
aacccagggaucauuaauc

SEQ ID NO: 139 - RNA, 19 nt, Artificial Sequence
  the sequence is synthesized
uaagcaaaacccagggauc

SEQ ID NO: 140 - RNA, 19 nt, Artificial Sequence
  the sequence is synthesized
ugcauuaagcaaaacccag

SEQ ID NO: 141 - RNA, 19 nt, Artificial Sequence
  the sequence is synthesized
ugaaccaagaugcauuaag

SEQ ID NO: 142 - RNA, 19 nt, Artificial Sequence
  the sequence is synthesized
aggaguugaaccaagaugc

SEQ ID NO: 143 - RNA, 19 nt, Artificial Sequence
  the sequence is synthesized
aggaaggaguugaaccaag

SEQ ID NO: 144 - RNA, 19 nt, Artificial Sequence
  the sequence is synthesized
ugugaggaaggaguugaac

SEQ ID NO: 145 - RNA, 19 nt, Artificial Sequence
  the sequence is synthesized
ugcaugugugaggaaggag

SEQ ID NO: 146 - RNA, 25 nt, Artificial Sequence
  the sequence is synthesized
uuucggaucuuccccauauucuuuc

SEQ ID NO: 147 - RNA, 25 nt, Artificial Sequence
  the sequence is synthesized
aaaacccagggaucauuaaucaggc

SEQ ID NO: 148 - RNA, 25 nt, Artificial Sequence
  the sequence is synthesized
aggaguugaaccaagaugcauuaag

SEQ ID NO: 149 - RNA, 25 nt, Artificial Sequence
  the sequence is synthesized
ugaggaaggaguugaaccaagaugc

SEQ ID NO: 150 - RNA, 25 nt, Artificial Sequence
  the sequence is synthesized
ugugugaggaaggaguugaaccaag

SEQ ID NO: 151 - RNA, 25 nt, Artificial Sequence
  the sequence is synthesized
ugcaugugugaggaaggaguugaac

SEQ ID NO: 152 - RNA, 19 nt, Artificial Sequence
  the sequence is synthesized
ucaggcacuccucaauugc

SEQ ID NO: 153 - RNA, 19 nt, Artificial Sequence
  the sequence is synthesized
uccgagagcucgaagacuc

SEQ ID NO: 154 - RNA, 19 nt, Artificial Sequence
  the sequence is synthesized
uucguccgagagcucgaag

SEQ ID NO: 155 - RNA, 19 nt, Artificial Sequence
  the sequence is synthesized
cuuuucguccgagagcucg

SEQ ID NO: 156 - RNA, 19 nt, Artificial Sequence
  the sequence is synthesized
ugccuuuucguccgagagc

SEQ ID NO: 157 - RNA, 19 nt, Artificial Sequence
  the sequence is synthesized
cguugccuuuucguccgag

SEQ ID NO: 158 - RNA, 19 nt, Artificial Sequence
  the sequence is synthesized
uucguugccuuuucguccg

SEQ ID NO: 159 - RNA, 19 nt, Artificial Sequence
  the sequence is synthesized
cggguucguugccuuuucg

SEQ ID NO: 160 - RNA, 19 nt, Artificial Sequence
  the sequence is synthesized
ucggguucguugccuuuuc

SEQ ID NO: 161 - RNA, 19 nt, Artificial Sequence
  the sequence is synthesized
ucauuacucaugucaaagg

SEQ ID NO: 162 - RNA, 19 nt, Artificial Sequence
  the sequence is synthesized
uugucuccgaagaaauaag

SEQ ID NO: 163 - RNA, 25 nt, Artificial Sequence
  the sequence is synthesized
uuuucguccgagagcucgaagacuc

SEQ ID NO: 164 - RNA, 25 nt, Artificial Sequence
  the sequence is synthesized
cacgaucggguucguugccuuuucg

SEQ ID NO: 165 - RNA, 25 nt, Artificial Sequence
  the sequence is synthesized
gcacgaucggguucguugccuuuuc

SEQ ID NO: 166 - RNA, 25 nt, Artificial Sequence
  the sequence is synthesized
ucugcauugucuccgaagaaauaag

SEQ ID NO: 167 - RNA, 19 nt, Artificial Sequence
  the sequence is synthesized
ggaucuuauuucuucggag

SEQ ID NO: 168 - RNA, 19 nt, Artificial Sequence
  the sequence is synthesized
agcaccgucuggccaagac

SEQ ID NO: 169 - RNA, 19 nt, Artificial Sequence
  the sequence is synthesized
aacucagcaccgucuggcc

SEQ ID NO: 170 - RNA, 19 nt, Artificial Sequence
  the sequence is synthesized
auggcaacucagcaccguc

SEQ ID NO: 171 - RNA, 25 nt, Artificial Sequence
  the sequence is synthesized
aauggcaacucagcaccgucuggcc

SEQ ID NO: 172 - RNA, 19 nt, Artificial Sequence
  the sequence is synthesized
ucgaccucgguuagaagac

SEQ ID NO: 173 - RNA, 19 nt, Artificial Sequence
  the sequence is synthesized
uuucgaccucgguuagaag

SEQ ID NO: 174 - RNA, 19 nt, Artificial Sequence
  the sequence is synthesized
acguuucgaccucgguuag

SEQ ID NO: 175 - RNA, 19 nt, Artificial Sequence
  the sequence is synthesized
acguacguuucgaccucgg

SEQ ID NO: 176 - RNA, 19 nt, Artificial Sequence
  the sequence is synthesized
aacguacguuucgaccucg

SEQ ID NO: 177 - RNA, 19 nt, Artificial Sequence
  the sequence is synthesized
gcgaucucggcuuugaggg

SEQ ID NO: 178 - RNA, 19 nt, Artificial Sequence
  the sequence is synthesized
auuggucuugucuuuagcc

SEQ ID NO: 179 - RNA, 19 nt, Artificial Sequence
  the sequence is synthesized
acugggcacggugagcgug

SEQ ID NO: 180 - RNA, 19 nt, Artificial Sequence
  the sequence is synthesized
ucacugggcacggugagcg

SEQ ID NO: 181 - RNA, 19 nt, Artificial Sequence
  the sequence is synthesized
uccucgcucacugggcacg

SEQ ID NO: 182 - RNA, 19 nt, Artificial Sequence
  the sequence is synthesized
ugcaguccucgcucacugg

SEQ ID NO: 183 - RNA, 19 nt, Artificial Sequence
  the sequence is synthesized
uacgcugcaguccucgcuc

SEQ ID NO: 184 - RNA, 19 nt, Artificial Sequence
  the sequence is synthesized
agcgucuacgcugcagucc

SEQ ID NO: 185 - RNA, 25 nt, Artificial Sequence
  the sequence is synthesized
uacguuucgaccucgguuagaagac

SEQ ID NO: 186 - RNA, 25 nt, Artificial Sequence
  the sequence is synthesized
ucgcucacugggcacggugagcgug

SEQ ID NO: 187 - RNA, 25 nt, Artificial Sequence
  the sequence is synthesized
uccucgcucacugggcacggugagc

SEQ ID NO: 188 - RNA, 25 nt, Artificial Sequence
  the sequence is synthesized
ugcaguccucgcucacugggcacgg

SEQ ID NO: 189 - RNA, 25 nt, Artificial Sequence
  the sequence is synthesized
uacgcugcaguccucgcucacuggg

SEQ ID NO: 190 - RNA, 25 nt, Artificial Sequence
  the sequence is synthesized
ucuacgcugcaguccucgcucacug

SEQ ID NO: 191 - RNA, 25 nt, Artificial Sequence
  the sequence is synthesized
uggacaaagcgucuacgcugcaguc

SEQ ID NO: 192 - RNA, 19 nt, Artificial Sequence
  the sequence is synthesized
ucuucuaaccgaggucgaa

SEQ ID NO: 193 - RNA, 25 nt, Artificial Sequence
  the sequence is synthesized
cccagugagcgaggacugcagcgua

SEQ ID NO: 194 - RNA, 19 nt, Artificial Sequence
  the sequence is synthesized
acagcagaaugcuguggau

SEQ ID NO: 195 - RNA, 25 nt, Artificial Sequence
  the sequence is synthesized
acccugcucagccugcacaaggugg

SEQ ID NO: 196 - RNA, 25 nt, Artificial Sequence
  the sequence is synthesized
cccugcucagccugcacaaggugga

SEQ ID NO: 197 - RNA, 25 nt, Artificial Sequence
  the sequence is synthesized
cuguccugcagcuccucccucugcc

SEQ ID NO: 198 - RNA, 25 nt, Artificial Sequence
  the sequence is synthesized
uguccugcagcuccucccucugcca

SEQ ID NO: 199 - RNA, 25 nt, Artificial Sequence
  the sequence is synthesized
uccugcagcuccucccucugccagc

SEQ ID NO: 200 - RNA, 25 nt, Artificial Sequence
  the sequence is synthesized
ccugcagcuccucccucugccagcg

SEQ ID NO: 201 - RNA, 25 nt, Artificial Sequence
  the sequence is synthesized
cugcagcuccucccucugccagcgc

SEQ ID NO: 202 - RNA, 25 nt, Artificial Sequence
  the sequence is synthesized
ugcagcuccucccucugccagcgcu

SEQ ID NO: 203 - RNA, 25 nt, Artificial Sequence
  the sequence is synthesized
gcagcuccucccucugccagcgcug

SEQ ID NO: 204 - RNA, 25 nt, Artificial Sequence
  the sequence is synthesized
cagcuccucccucugccagcgcugu

SEQ ID NO: 205 - RNA, 25 nt, Artificial Sequence
  the sequence is synthesized
agcuccucccucugccagcgcugug

SEQ ID NO: 206 - RNA, 25 nt, Artificial Sequence
  the sequence is synthesized
uccucccucugccagcgcugugaca

SEQ ID NO: 207 - RNA, 25 nt, Artificial Sequence
  the sequence is synthesized
ccaccuugugcaggcugagcagggu

SEQ ID NO: 208 - RNA, 25 nt, Artificial Sequence
  the sequence is synthesized
uccaccuugugcaggcugagcaggg

SEQ ID NO: 209 - RNA, 25 nt, Artificial Sequence
  the sequence is synthesized
ggcagagggaggagcugcaggacag

SEQ ID NO: 210 - RNA, 25 nt, Artificial Sequence
  the sequence is synthesized
uggcagagggaggagcugcaggaca

SEQ ID NO: 211 - RNA, 25 nt, Artificial Sequence
  the sequence is synthesized
gcuggcagagggaggagcugcagga

SEQ ID NO: 212 - RNA, 25 nt, Artificial Sequence
  the sequence is synthesized
cgcuggcagagggaggagcugcagg

SEQ ID NO: 213 - RNA, 25 nt, Artificial Sequence
  the sequence is synthesized
gcgcuggcagagggaggagcugcag

SEQ ID NO: 214 - RNA, 25 nt, Artificial Sequence
  the sequence is synthesized
agcgcuggcagagggaggagcugca

SEQ ID NO: 215 - RNA, 25 nt, Artificial Sequence
  the sequence is synthesized
cagcgcuggcagagggaggagcugc

SEQ ID NO: 216 - RNA, 25 nt, Artificial Sequence
  the sequence is synthesized
acagcgcuggcagagggaggagcug

SEQ ID NO: 217 - RNA, 25 nt, Artificial Sequence
  the sequence is synthesized
cacagcgcuggcagagggaggagcu

SEQ ID NO: 218 - RNA, 25 nt, Artificial Sequence
  the sequence is synthesized
ugucacagcgcuggcagagggagga

SEQ ID NO: 219 - RNA, 25 nt, Artificial Sequence
  the sequence is synthesized
ccaaggaccuauaugugguagagua

SEQ ID NO: 220 - RNA, 25 nt, Artificial Sequence
  the sequence is synthesized
cgacuacaagcgaauuacugugaaa

SEQ ID NO: 221 - RNA, 25 nt, Artificial Sequence
  the sequence is synthesized
ccucugaacaugaacugacauguca

SEQ ID NO: 222 - RNA, 25 nt, Artificial Sequence
  the sequence is synthesized
ccaaaugaaaggacucacuugguaa

SEQ ID NO: 223 - RNA, 25 nt, Artificial Sequence
  the sequence is synthesized
gcacugacauucaucuuccguuuaa

SEQ ID NO: 224 - RNA, 25 nt, Artificial Sequence
  the sequence is synthesized
gggauccauccuguuguuccucauu

SEQ ID NO: 225 - RNA, 25 nt, Artificial Sequence
  the sequence is synthesized
uccaugaagugucaugaaucuuguu

SEQ ID NO: 226 - RNA, 25 nt, Artificial Sequence
  the sequence is synthesized
cccucugaucgucgauuggcagcuu

SEQ ID NO: 227 - RNA, 25 nt, Artificial Sequence
  the sequence is synthesized
aaggaagaugagcaagugauucagu

SEQ ID NO: 228 - RNA, 25 nt, Artificial Sequence
  the sequence is synthesized
cagcuaaccucuguuauccucacuu

SEQ ID NO: 229 - RNA, 25 nt, Artificial Sequence
  the sequence is synthesized
caggcucuugcugucugacucaaau

SEQ ID NO: 230 - RNA, 25 nt, Artificial Sequence
  the sequence is synthesized
gacgcaggcguuuacugcugcauaa

SEQ ID NO: 231 - RNA, 25 nt, Artificial Sequence
  the sequence is synthesized
uacucuaccacauauagguccuugg

SEQ ID NO: 232 - RNA, 25 nt, Artificial Sequence
  the sequence is synthesized
uuucacaguaauucgcuuguagucg

SEQ ID NO: 233 - RNA, 25 nt, Artificial Sequence
  the sequence is synthesized
ugacaugucaguucauguucagagg

SEQ ID NO: 234 - RNA, 25 nt, Artificial Sequence
  the sequence is synthesized
uuaccaagugaguccuuucauuugg

SEQ ID NO: 235 - RNA, 25 nt, Artificial Sequence
  the sequence is synthesized
uuaaacggaagaugaaugucagugc

SEQ ID NO: 236 - RNA, 25 nt, Artificial Sequence
  the sequence is synthesized
aaugaggaacaacaggauggauccc

SEQ ID NO: 237 - RNA, 25 nt, Artificial Sequence
  the sequence is synthesized
aacaagauucaugacacuucaugga

SEQ ID NO: 238 - RNA, 25 nt, Artificial Sequence
  the sequence is synthesized
aagcugccaaucgacgaucagaggg

SEQ ID NO: 239 - RNA, 25 nt, Artificial Sequence
  the sequence is synthesized
acugaaucacuugcucaucuuccuu

SEQ ID NO: 240 - RNA, 25 nt, Artificial Sequence
  the sequence is synthesized
aagugaggauaacagagguuagcug

SEQ ID NO: 241 - RNA, 25 nt, Artificial Sequence
  the sequence is synthesized
auuugagucagacagcaagagccug

SEQ ID NO: 242 - RNA, 25 nt, Artificial Sequence
  the sequence is synthesized
uuaugcagcaguaaacgccugcguc